(19) United States
(12) Patent Application Publication
(10) Pub. No.: US 2022/0288048 A1
(43) Pub. Date: Sep. 15, 2022

(54) PIMAVANSERIN FOR TREATING SCHIZOPHRENIA OR FOR TREATING PSYCHOSIS SECONDARY TO NEURODEGENERATIVE DISORDERS OR DEPRESSIVE DISORDER

(71) Applicant: ACADIA Pharmaceuticals Inc., San Diego, CA (US)

(72) Inventors: Dragana Bugarski Kirola, Atlanta, GA (US); Ethan S. Burstein, San Diego, CA (US)

(21) Appl. No.: 17/629,098

(22) PCT Filed: Jul. 22, 2020
(86) PCT No.: PCT/US2020/043103

§ 371 Date: Jan. 21, 2022

Related U.S. Application Data
(60) Provisional application No. 62/963,736, filed on Jan. 21, 2020.
(60) Provisional application No. 62/939,250, filed on Nov. 22, 2019.
(60) Provisional application No. 62/936,824, filed on Nov. 18, 2019.
(60) Provisional application No. 62/876,896, filed on Jul. 22, 2019.

(51) Int. Cl. A61K 31/4468; A61P 25/18

(52) U.S. Cl. CPC A61K 31/4468; A61P 25/18

(57) ABSTRACT

The present disclosure relates generally to therapeutic use of pimavanserin or a pharmaceutical acceptable salt thereof. More specifically, the present disclosure provides methods for treating schizophrenia by administering pimavanserin or a pharmaceutical acceptable salt thereof as an adjunct therapy in a patient who has an inadequate response to another antipsychotic therapy.

### CROSS-REFERENCE TO RELATED APPLICATIONS

This application claims benefit of an priority to U.S. Provisional Patent Application No. 62/876,896, filed Jul. 22, 2019; U.S. Provisional Application No. 62/936,824, filed Nov. 18, 2019; U.S. Provisional Application No. 62/939,250, filed Nov. 22, 2019; and U.S. Provisional Application No. 62/963,736, filed Jan. 21, 2020, the entire contents of each of which are hereby incorporated by reference.

### BACKGROUND

Approximately 1% of adults in the U.S. suffer from schizophrenia, a debilitating and lifelong condition. See, e.g., NAMI, Mental Help, PsyCom, SAMHSA study, NIMH data consolidation. According to the American Psychiatric Association, 30% of schizophrenia patients (˜700,000) have an inadequate response to their antipsychotic therapy. Approximately 40-50% of schizophrenia patients (˜1 million) experience prominent negative symptoms, such as reduced expression of emotions, reduced feelings of pleasure in everyday life, difficulty beginning and sustaining activities, and reduced speaking. There are no approved treatments to date for negative symptoms of schizophrenia despite a high unmet need.

Pimavanserin (formerly ACP-103) is a potent and selective 5-hydroxytryptamine (5-HT)2A receptor inverse agonist of interest as therapeutic for neuropsychiatric diseases and disorders, such as, for example, Parkinson's disease psychosis, sleep disorders, and schizophrenia. See, e.g., U.S. Pat. No. 7,601,740 B2; Vanover et al., The Journal of Pharmacology and Experimental Therapeutics, 2006, vol. 317, no. 2, pages 910-918. Preparations of pimavanserin and pimavanserin in salt and crystalline forms have been described in, for instance, WO 2006/037043 A1, WO 2006/036874 A1, WO 2007/133802 and WO 2008/144326.

Tolerability and safety of pimavanserin has been studied in healthy volunteers, see, e.g., Vanover et al., The Journal of Clinical Pharmacology, 2007, vol. 47, no. 6, pages 704-714, and clinical studies with pimavanserin have been undertaken.

A demand exists for adjunctive treatments for schizophrenia, and in particular, for the predominant negative symptoms of schizophrenia.

### SUMMARY

The present disclosure relates in part to the discovery that the administration of pimavanserin can treat the loss of normal functioning in a patient suffering from schizophrenia, for example, in a patient who has a partial but inadequate response to another antipsychotic treatment and/or in a patient who is or has concurrently taking another antipsychotic agent.

Accordingly, in one aspect, the disclosure relates to a method of treating the loss of normal functions in a patient suffering from schizophrenia, wherein the patient has had a partial but inadequate response to an antipsychotic treatment, and is being administering the antipsychotic treatment, comprising orally administering once daily an effective amount of pimavanserin to the patient. In certain embodiments, the patient is not resistant to antipsychotic treatment.

In certain embodiments, the loss of normal function is a negative symptom of schizophrenia, e.g. a prominent negative symptom (or e.g., a pre-dominant negative symptom), e.g., at least one of: emotional blunting, emotional withdrawal, poor rapport, passive/apathetic social withdrawal, difficulty in abstract thinking, lack of spontaneity and flow of conversation, and stereotyped thinking. In certain embodiments, the loss of normal function is an aspect of sleep disturbance associated with schizophrenia.

In certain embodiments, after 6 weeks of administering pimavanserin, the patient improves on the negative syndrome subscale of PANSS compared to baseline. In certain embodiments, after 6 weeks of administering pimavanserin, the patient improves on the PANSS Marder Negative factor score compared to baseline. In certain embodiments, after 6 weeks of administering pimavanserin, the patient improves on the Karolinska Sleepiness Scale compared to baseline. In certain embodiments, after 26 weeks of administering pimavanserin, the patient improves on the PANSS Marder Negative factor score compared to baseline. In certain embodiments, after 26 weeks of administering pimavanserin, the patient improves on the Karolinska Sleepiness Scale compared to baseline. In some embodiments, after 26 weeks of administering pimavanserin, the patient improves on the Negative Symptom Assessment-16 total score as compared to baseline. In other embodiments, after 26 weeks of administering pimavanserin, the patient improves on the Personal and Social Performance Scale (PSP) score as compared to baseline. In certain embodiments, after 26 weeks of administering pimavanserin, the patient improves on the Clinical Global Impression of Schizophrenia Scale-Severity (CGI-SCH-S) for the negative symptoms of schizophrenia score, the Positive and Negative Syndrome Scale (PANSS) score, the Brief Assessment of Cognition in Schizophrenia (BACS) score, or the 10-item Drug Attitude Inventory (DAI-10) score as compared to baseline.

Also provided herein is a method of treating a patient having a partial but inadequate response to an antipsychotic treatment for schizophrenia, comprising: continuing to administer an effective amount of the antipsychotic; and administering an effective amount of pimavanserin, thereby treating loss of normal functions in the patient suffering from schizophrenia. In some embodiments, the loss of normal function is a negative symptom of schizophrenia.

In another aspect, the disclosure relates to a method of treating or diminishing a negative symptom of schizophrenia in a patient having a partial but inadequate response to an antipsychotic alone, comprising administering an effective amount of pimavanserin to the patient and continuing administration of the antipsychotic. In certain embodiments, the antipsychotic is selected from the group consisting of quetiapine, clozapine, aripiprazole, asenapine, cariprazine, brexpiprazole, lurasidone, olanzapine, and risperidone, and/or long-acting formulations thereof, including long acting aripiprazole or risperidone.

In certain embodiments, the method comprises administering about 10 mg to about 34 mg daily pimavanserin. In certain embodiments, the method comprises administering daily 10 mg, 20 mg or 34 mg pimavanserin. In certain embodiments, administering an effective amount of pimavanserin comprises: administering about 20 mg of pimavanserin daily for one to three weeks; and then administering about 10 mg or about 34 mg pimavanserin daily.

Provided herein, in part, is a method of treating or diminishing a negative symptom of schizophrenia in a patient being administered an antipsychotic, comprising additionally administering an effective amount of pimavanserin to the patient and continuing administration of the antipsychotic. The negative symptom may be at least one of: emotional blunting, emotional withdrawal, poor rapport, passive/apathetic social withdrawal, difficulty in abstract thinking, lack of spontaneity and flow of conversation, and stereotyped thinking. In some embodiment, after 6 weeks of administering pimavanserin, the patient improves on the negative syndrome subscale of PANSS compared to baseline. In other embodiments, after 6 weeks of administering pirnavanserin, the patient improves on the PANSS Marder Negative factor score compared to baseline. In certain embodiments, after 6 weeks of administering pimavanserin, the patient improves on the Karolinska Sleepiness Scale.

In some embodiments, wherein after 26 weeks of administering pimavanserin, the patient improves on the PANSS Marder Negative factor score compared to baseline. In other embodiments, after 26 weeks of administering pimavanserin, the patient improves on the Karolinska Sleepiness Scale. In certain embodiments, after 26 weeks of administering pimavanserin, the patient improves on the Negative Symptom Assessment-16 total score as compared to baseline. In some embodiments, after 26 weeks of administering pimavanserin, the patient improves on the Personal and Social Performance Scale (PSP) score as compared to baseline. In other embodiments, after 26 weeks of administering pimavanserin, the patient improves on the Negative Symptom Assessment (NSA-16) total score, Clinical Global Impression of Schizophrenia Scale-Severity (CGI-SCH-S) for the negative symptoms of schizophrenia score, the Positive and Negative Syndrome Scale (PANSS) score, the Brief Assessment of Cognition in Schizophrenia (BACS) score, or the 10-item Drug Attitude Inventory (DAI-10) score as compared to baseline.

In another aspect, the present disclosure provides a method of adjunctively treating negative symptoms of schizophrenia in need thereof, wherein the patient is currently being administered an antipsychotic, comprising additionally administering an effective amount of pimavanserin daily, wherein after 26 weeks of administration, the patient demonstrates a statistically significant reduction on the Negative Symptom Assessment-16 (NSA-16) total score. In some embodiments, the effective amount is 10 mg to 34 mg of pimavanserin daily. In certain embodiments, the effective amount is 34 mg of pimavanserin daily. In other embodiments, the effective amount is 10 or 20 mg of pimavanserin daily. In some embodiments, administering comprises administering an initial dose of 20 mg daily of pimavanserin for 1 day, 1 week, or 1-8 weeks, and then administering 34 mg daily for at least 18 weeks. In other embodiments, the antipsychotic is one of: aripiprazole, asenapine, cariprazine, brexpiprazole, lurasidone, olanzapine or risperidone.

In another aspect, a method of treating psychosis secondary to neurodegenerative disorders or major depressive disorder, in a patient in need thereof, comprising administering about 20 mg of pimavanserin daily for one to three weeks or more; and then administering about 10 mg or about 34 mg pimavanserin daily is provided herein. In some embodiments, psychosis secondary to neurodegenerative disorders is dementia related psychosis.

### DESCRIPTION OF THE DRAWINGS

FIG. 1 is a graph showing the average change in PANSS Total Score from baseline on a weekly basis, for the number of subjects indicated.

FIG. 2 is a graph showing the average change in CGI-S score from baseline on a weekly basis, for the number of subjects indicated.

FIG. 3 is a graph showing the average change in PANSS score from baseline on a weekly basis, for the subset of European patients.

FIG. 4 is a graph showing the average change in PANSS Negative Symptom Scale from baseline on a weekly basis, for the number of subjects indicated.

FIG. 5 is a graph showing the effect of risperidone and pimavanserin on scPCP-induced deficit in social interaction behaviors in the 10-min social interaction test conducted in scPCP-treated C57B16 male mice.

FIG. 6 is a graph showing the effect of atypical APDs (antipsychotic drugs) and pimavanserin on scPCP and ARS (acute restraint stress)-induced deficits in C57B16 male mice in social interaction behaviors in the 10-min social interaction test.

FIG. 7 is a graph showing the effect of a co-administration of atypical APDs and pimavanserin on scPCP and ARS-treated C57B16 male mice on social interaction behaviors in the 10-min social interaction test.

FIG. 8 is a graph showing the effect of double-dose risperidone, risperidone, pimavanserin, and risperidone combined with pimavanserin on scPCP and ARS-treated C57B16 male mice on social interaction behaviors in the 10-min social interaction test.

FIG. 9 shows the Primary End point: NSA-16 Total Score results after 26 weeks of administration.

FIG. 10 shows the NSA—16 Total Score—34 mg patients.

FIG. 11 shows mean change from baseline among pre-specified subgroup of patients enrolled in Europe for PANSS Total Score (FAS Population).

FIG. 12 shows mean change from baseline among pre-specified subgroup of patients enrolled in Europe for CGI-S score (FAS Population).

FIG. 13 shows mean change from baseline among patients enrolled in Europe for PANSS Marder Factor Score for negative symptoms (FAS Population).

FIG. 14 shows mean change from baseline among patients enrolled in Europe for PANSS Marder Factor Score for positive symptoms (FAS Population).

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

“Treating” includes any effect, e.g., diminishing, lessening, reducing, modulating, or eliminating, that results in the improvement of the condition, disease, disorder and the like.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the disclosure can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

In the present specification, the term “therapeutically effective amount” means the amount of the subject compound that will elicit the biological or medical response of a tissue, system, animal or human that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect, such as an amount which results in the treatment of depression.

In some embodiments, a patient taking an anti-psychotic to treat schizophrenia is administered with pimavanserin or a pharmaceutically acceptable salt thereof, to treat the schizophrenia.

The term “pharmaceutically acceptable salt” refers to a salt of a compound that does not cause significant irritation to an organism to which it is administered and does not abrogate the biological activity and/or properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid.

“Clinical Global Impression (CGI-S)” Severity scale is a clinician-rated, 7-point scale that is designed to rate the severity of the subject at the time of assessment using the Investigator's judgment and past experience with subjects who have the same disorder (i.e., schizophrenia). “Clinical Global Impression-Improvement (CGI-I)” is a clinician-rated, 7-point scale that is designed to rate the improvement in the subject's schizophrenia at the time of assessment, relative to the symptoms at Baseline.

“Karolinska Sleepiness Scale (KSS)” is a scale for evaluating subjective sleepiness (Kaida et al. Clinical Neurophysiology 2006, 117, 7, 1574-1581).

“Positive and Negative Symptom Scale (PANSS)” is a 30-item, 7-point rating system that was developed to cover a range of negative symptoms not adequately measured by the Brief Psychiatric Rating Scale. It has sections that specifically measure (1) positive symptoms (delusions, conceptual disorganization, hallucinations, excitement, grandiosity, suspiciousness/persecution, hostility), (2) negative symptoms (blunted affect, emotional withdrawal, poor rapport, passive/apathetic social withdrawal, difficulty in abstract thinking, lack of spontaneity and flow of conversation, stereotyped thinking), and (3) general psychopathology (somatic concern, anxiety, guilt feelings, tension, mannerisms and posturing, depression, motor retardation, uncooperativeness, unusual thought content, disorientation, poor attention, lack of judgment and insight, disturbance of volition, poor impulse control, preoccupation, active social avoidance) in schizophrenic subjects. The PANSS is widely used in trials of antipsychotic drug treatment, and has been formally validated for such use.

“Marder PANSS factors” is a subset of 5 of the PANSS factors: positive symptoms, negative symptoms, disorganized thinking, and the associated symptoms of hostility/excitement and depression/anxiety.

The “Clinical Global Impression Scale (CGI)” is a clinician rated scale: the CGI-Severity (CGI-S), rates illness severity, and CGI-Improvement (CGI-I), rates change from the baseline of treatment.

The “Personal and Social Performance Scale (PSP)” measures a patients' degree of psychosocial functioning in four areas: socially useful activities including work and study, personal and social relationships, self-care, and disturbing and aggressive behavior.

The “drug attitude inventory (DAI-10)” is a self-reporting measure reflecting a patient's attitude toward psychiatric medication, with a scoring range between −10 and 10. A total score greater than 0 indicates a positive attitude toward psychiatric medications and a total score less than 0 indicates a negative attitude toward psychiatric medications.

The “Karolinska Sleepiness Scale (KSS)” is a scale for evaluating subjective sleepiness (Kaida et al. Clinical Neurophysiology 2006, 117, 7, 1574-1581). It can be helpful in assessing the effects of drugs. The KSS is a nine point scale (1=extremely alert to 9=extremely sleepy-fighting sleep).

The “Calgary Depression Scale for Schizophrenia (CDSS)” is a 9-item, 4-point scale that was specifically designed to measure depressive symptoms in schizophrenia, separate from the positive, negative, and extrapyramidal symptoms observed in this population. It has been widely used in treatment trials in schizophrenia and has been validated for such use.

The “Negative Symptom Assessment-16 (NSA-16)” is a 16-item scale used for assessing the presence, severity, and range of the negative symptoms of schizophrenia. The NSA-16 assesses five domains of negative symptoms of schizophrenia: (1) communication, (2) emotion/affect, (3) social involvement, (4) motivation, and (5) retardation.

As used herein, a patient with schizophrenia having a “partial but inadequate response” to an antipsychotic refers to a patient who has been or is taking an antipsychotic which has partially, but not completely, treated the loss of normal functions in the patient.

### Pimavanserin

N-(4-Fluorophenylmethyl)-N-(1-methylpiperidin-4-yl)-N′-(4-(2-methylpropyloxy)phenylmethyl)carbamide is also known as pimavanserin; N-[(4-fluorophenyl)methyl]-N-(1-methyl-4-piperidinyl)-N′-[[4-(2-methylpropoxy)phenyl]methyl]-urea, 1-(4-fluorobenzyl)-1-(1-methylpiperidin-4-yl)-3-[4-(2-methylpropoxy)benzyl]urea or ACP-103 and is represented by the chemical formula:

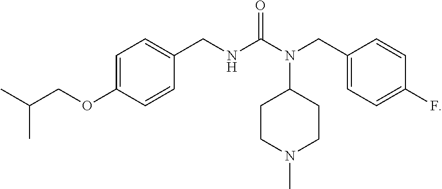 [SMILES: CC(C)COc1ccc(CNC(=O)N(Cc2ccc(F)cc2)C2CCN(C)CC2)cc1]

N-(4-Fluorophenylmethyl)-N-(1-methylpiperidin-4-yl)-N′-(4-(2-methylpropyloxy)phenylmethyl)carbamide maybe administered as a tartrate salt, which is urea, N-[(4-fluorophenyl)methyl]-N-(1-methyl-4-piperidinyl)-N′-[[4-(2-methylpropoxy)phenyl]methyl]-,(2R,3R)-2,3-dihydroxybutanedioate (2:1), and represented by the chemical formula:

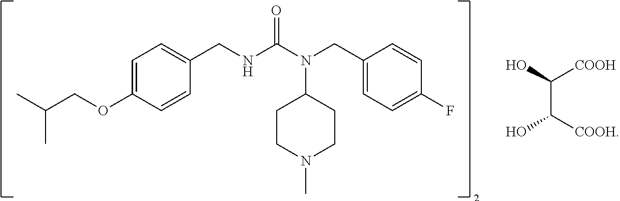

Pimavanserin (i.e., N-(4-Fluorophenylmethyl)-N-(1-methylpiperidin-4-yl)-N′-(4-(2-methylpropyloxy)phenylmethyl)carbamide) can be synthesized according to the method disclosed in Scheme I.

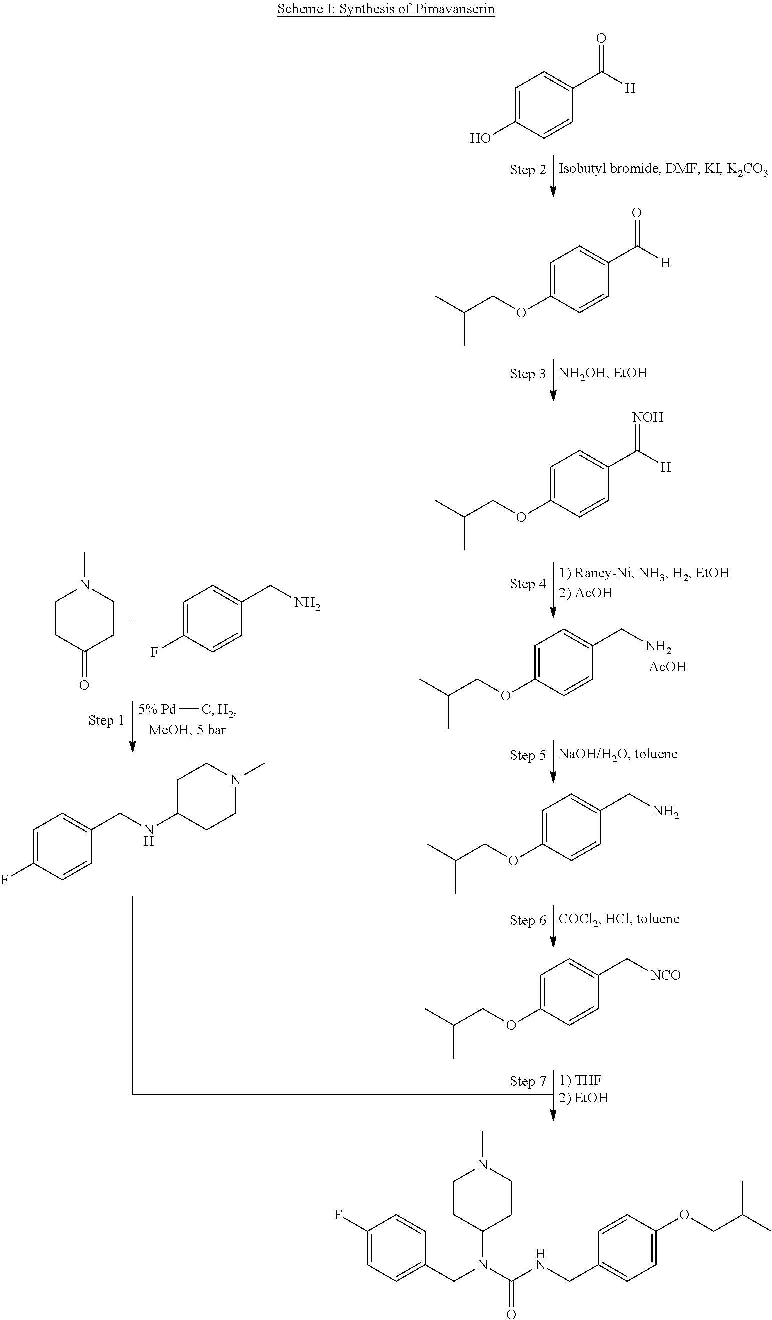

Pimavanserin can be obtained in a number of salt and crystalline forms. Exemplary pharmaceutically acceptable salts include the tartrate, hemi-tartrate, citrate, fumarate, maleate, malate, phosphate, succinate, sulphate, and edisylate (ethanedisulfonate) salts. Pimavanserin, and salts thereof including the aforementioned ions, among others, are described, for example, in U.S. Pat. Nos. 7,790,899; 7,868,176; and 7,923,564, and International Patent Application WO2017/015272, the entirety of which is incorporated herein by reference. In an embodiment provided herein, pimavanserin is the tartrate salt of pimavanserin.

Several crystalline forms of the tartrate salt are referred to as crystalline Form A, Form B, Form C, Form D, Form E and Form F, and are described in U.S. Pat. No. 7,732,615, which is incorporated herein by reference in its entirety. In one embodiment, the crystalline form of the tartrate salt of pimavanserin is Form C, which exhibits an X-ray powder diffraction pattern comprising peaks having d-values in angstroms (Å) of about 10.7, about 4.84, about 4.57, and about 3.77. Specifically the X-ray powder diffraction pattern of Form C exhibits the following characteristic peaks expressed in d-values (A): 12.0 (w), 10.7 (vs), 7.4 (vw), 6.9 (vw), 6.6 (vw), 6.2 (w), 5.86 (m), 5.53 (w), 5.28 (m), 5.16 (m), 4.84 (vs), 4.70 (m), 4.57 (s), 4.38 (m), 4.09 (w), 3.94 (w), 3.77 (s), 3.71 (m), 3.49 (w), 3.46 (w), 3.25 (w), 3.08 (w), and 2.93 (w). In various embodiments, Form C is present in a solid form of pimavanserin in amounts of at least about 50%, 70%, 80%, 90%, 95%, or 98%, with the remainder being other crystalline forms (including hydrates and solvates) and/or amorphous forms.

### Formulation and Dosage

Pimavanserin (including, for example, the tartrate salt) may be formulated into tablets, such as is described in more detail in U.S. Pat. No. 7,790,899, and U.S. Patent Publication No. 2007-0264330, filed May 15, 2007, each entitled “PHARMACEUTICAL FORMULATIONS OF PIMAVANSERIN,” which are incorporated herein by reference in their entireties.

In some embodiments, the drug product is formulated with standard pharmaceutical excipients at a 34 mg pimavanserin (40 mg of pimavanserin tartrate) in capsules for oral administration. In certain embodiments the capsule formulation includes inactive ingredients magnesium stearate and microcrystalline cellulose. The following inactive ingredients can, for example, be present as components of the capsule shell: black iron oxide, FD&C blue #1, hypromellose, titanium dioxide, and yellow iron oxide.

In certain embodiments, a capsule or tablet can contain 40 mg of pimavanserin tartrate, which is equivalent to 34 mg of pimavanserin free base, or 20 mg of pimavanserin tartrate, which is equivalent to 17 mg of pimavanserin free base, or 11.8 mg of pimavanserin tartrate, which is equivalent to 10 mg of pimavanserin free base.

In some embodiments, the drug product is formulated with standard pharmaceutical excipients at a 17 mg strength (20 mg of pimavanserin tartrate) in immediate-release tablets for once-daily oral administration.

In some embodiments, the drug product is formulated with standard pharmaceutical excipients at a 10 mg strength (11.8 mg of pimavanserin tartrate) in immediate-release tablets for once-daily or twice-daily oral administration.

In some embodiments, the dose for the indication of adjunctive treatment of schizophrenia is 34 mg pimavanserin taken orally as two 17 mg tablets once daily.

In some embodiments, the dose and pharmaceutical composition of pimavanserin are those disclosed in International Patent Publication No. WO2019/046167, which is incorporated herein for all purposes.

In some embodiments, the pharmaceutical composition comprises granulated pimavanserin tartrate, Form C which may include a small percentage of Form A, including a pharmaceutically acceptable diluent, binder or excipient, or combination thereof.

In some embodiments, the pharmaceutical compositions are provided as a two-piece hard shell capsules made of gelatin (fish, mammalian, or vegetable sourced) or other combinations. The two-piece hard shell capsules may contain the pimavanserin granules with a filler/diluent and/or lubricants. In some embodiments, the capsules are size 3 or 4 capsules. In some embodiments, the capsules are size 4 capsules. In some embodiment, the capsules are two-piece capsules of gelatin or hydroxypropyl methylcellulose (HPMC). Some commercial examples are VCAPS®, VCAPS® PLUS, CONI-SNAP® capsules marketed by Capsugel.

In some embodiments, the doses referred to herein refers to pimavanserin free base. In some embodiments, the doses referred to herein refers to pimavanserin tartrate Form C (e.g., 40 mg of pimavanserin tartrate, equivalent to 34 mg pimavanserin free base). In some embodiments, the doses refer to pimavanserin tartrate Form C encapsulated in capsules of size 3 or 4, such as capsules of size 4.

Provided are also embodiments wherein pimavanserin (granulated), microcrystalline cellulose, for example Avicel PH302 or an equivalent microcrystalline cellulose, and/or magnesium stearate, for example vegetable grade are encapsulated in a capsule of size 4, for example a two-piece capsule.

One embodiment of the compositions described herein includes pimavanserin tartrate granulation without binder, dried, and thereafter blended with less than 60% w/w microcrystalline cellulose such as Avicel PH302 or equivalent microcrystalline cellulose, and about 1% w/w magnesium stearate.

In some embodiments the compositions described herein comprise granulated pimavanserin and microcrystalline cellulose is at least 20% w/w microcrystalline cellulose, such as 30% w/w microcrystalline cellulose, such as 40% w/w microcrystalline cellulose, such as 50% w/w microcrystalline cellulose, such as 50-89% w/w microcrystalline cellulose, such as 20-94% w/w, such as 50-94% w/w, such as 57-94% w/w, such as 57-89% w/w microcrystalline cellulose, such as 57-79% w/w microcrystalline cellulose, or 57-60% w/w microcrystalline cellulose, or 57-59.5% w/w microcrystalline cellulose, or 58.5-59.5% w/w microcrystalline cellulose, or 59% w/w microcrystalline cellulose.

In some embodiments the compositions described herein comprise granulated pimavanserin and microcrystalline cellulose and magnesium stearate, such as 0.1-3% w/w, such as 0.5-2% w/w magnesium stearate, or 0.5-1.5% w/w magnesium stearate, or 1% w/w magnesium stearate.

In some embodiments the compositions described herein comprise granulated pimavanserin (5, 10, 20 or 34 mg) and microcrystalline cellulose is at least 20% w/w microcrystalline cellulose, such as 30% w/w microcrystalline cellulose, such as 40% w/w microcrystalline cellulose, such as 50% w/w microcrystalline cellulose, such as 50-89% w/w microcrystalline cellulose, such as 20-94% w/w, such as 50-94% w/w, such as 57-94% w/w, such as 57-89% w/w microcrystalline cellulose, such as 57-79% w/w microcrystalline cellulose, or 57-60% w/w microcrystalline cellulose, or 57-59.5% w/w microcrystalline cellulose, or 58.5-59.5% w/w microcrystalline cellulose, or 59% w/w microcrystalline cellulose and magnesium stearate, such as 0.1-3% w/w, such as 0.5-2% w/w magnesium stearate, or 0.5-1.5% w/w magnesium stearate, or 1% w/w magnesium stearate.

The compositions disclosed herein comprise pimavanserin and additional compatible excipients, e.g. sugars, sucrose, mannitol, sorbitol, polysaccharides (e.g. from corn, wheat, rice, potato), as well as pregelatinized or partially pregelatinized starches (e.g. STARCH 1500@), cellulose preparations such as microcrystalline cellulose (MCC) (e.g. AVICEL® PH 302, AVICEL®PH 102, VIVAPUR® 302, VIVAPUR® 102, EMCOCEL® HD 90), silicified microcrystalline cellulose (e.g. PROSOLV® 50, PROSOLV® 90, PROSOLV® HD90), lactose cellulose blends (e.g. CELLATOSE® 80, CELLATOSE® 90, PROSOLV® EASYtab SP), hydroxypropylmethyl cellulose, hydroxymethyl cellulose, polyvinylpyrrolidone, lubricants such as magnesium stearate, sodium stearyl fumarate, colloidal silicon dioxide, and talc.

Provided are also embodiments wherein 34 mg pimavanserin (granulated) (equivalent to 40 mg pimavanserin tartrate), microcrystalline cellulose, such as microcrystalline cellulose having a particle size distribution (D90) of 180-340 m, for example Avicel PH302 or an equivalent microcrystalline cellulose, and/or magnesium stearate, for example vegetable grade are encapsulated in a capsule of size 4, for example a two-piece capsule.

Provided are also embodiments wherein 10 or 20 mg pimavanserin (granulated), microcrystalline cellulose, for example Avicel PH302 or an equivalent microcrystalline cellulose, and/or magnesium stearate, for example vegetable grade are encapsulated in a capsule of size 4, for example a two-piece capsule.

Provided are also embodiments wherein 34 mg pimavanserin (granulated), 59 mg microcrystalline cellulose, for example Avicel PH302 or an equivalent microcrystalline cellulose, and/or 1 mg magnesium stearate, for example vegetable grade are encapsulated in a capsule of size 4, for example a two-piece capsule. No other excipients were added.

Also provided is a pharmaceutical composition, comprising a capsule of pimavanserin and one or more pharmaceutically acceptable excipient(s) as provided herein, wherein the composition is formulated such that at least 80% of pimavanserin is released in 30 minutes upon administration to a subject.

Also provided is a pharmaceutical composition, comprising a capsule of pimavanserin and one or more pharmaceutically acceptable excipient(s) as provided herein, wherein the composition is formulated such that at least 80% of the pimavanserin is released from the composition within 30 minutes upon in vitro dissolution testing according to USP <711> (apparatus 1 (basket apparatus)).

In some embodiments, a pharmaceutically acceptable salt of pimavanserin is administered to the patient. In some specific embodiments, a tartrate salt of pimavanserin is administered to the patient.

In some embodiments, the pharmaceutically acceptable salt of pimavanserin comprises an anion selected from the group consisting of phosphate, sulphate, nitrate, diphosphate, besylate, bicarbonate, carbonate, clavulanate, edisylate, isothionate, borate, halide including e.g., chloride and bromide, nitrate, acetate, succinate, lactate, lactobionate, laurate, mandelate, malate, citrate, fumarate, maleate, oleate, oxalate, ascorbate, nicotinate, benzoate, mesylate, salicylate, stearate, tannate, tosylate, valerate, methanesulfonate, ethanesulfonate, benzenesulfonate, p-toluensulfonate, 2-ethane disulfonate, and naphthalenesulfonate.

### Methods

In one aspect, the disclosure relates to a method of treating the loss of normal functions in a patient suffering from schizophrenia, wherein the patient has had a partial but inadequate response to an antipsychotic treatment, and is being administering the antipsychotic treatment, comprising orally administering once daily an effective amount of pimavanserin to the patient. In certain embodiments, the patient is not resistant to antipsychotic treatment. In certain embodiments, the antipsychotic is selected from the group consisting of quetiapine, clozapine, aripiprazole, asenapine, cariprazine, brexpiprazole, lurasidone, olanzapine, and risperidone.

In certain embodiments, the loss of normal function is a negative symptom of schizophrenia, e.g., at least one of: emotional blunting, emotional withdrawal, poor rapport, passive/apathetic social withdrawal, difficulty in abstract thinking, lack of spontaneity and flow of conversation, and stereotyped thinking. Measurement of a negative symptom of schizophrenia may be performed using a clinical scale such as the negative syndrome subscale of PANSS or the PANSS Marder Negative factor score. In certain embodiments, the loss of normal function is an aspect of sleep disturbance associated with schizophrenia. In certain embodiments, measurement of an aspect of sleep disturbance can be performed using the Karolinska Sleepiness Scale.

Upon administration of pimavanserin to a patient as described herein, a score on a clinical scale (e.g., the negative syndrome subscale of PANSS, the PANSS Marder Negative factor score, or KSS) improves. In certain embodiments, a score on a clinical scale improves after about 4 weeks, after about 5 weeks, or after about 6 weeks of administration (e.g., daily administration) of pimavanserin. In certain embodiments, after 6 weeks of administering pimavanserin, the patient improves on the negative syndrome subscale of PANSS compared to baseline. In certain embodiments, after 6 weeks of administering pimavanserin, the patient improves on the PANSS Marder Negative factor score compared to baseline. In certain embodiments, after 6 weeks of administering pimavanserin, the patient improves on the Karolinska Sleepiness Scale compared to baseline.

In another aspect, the disclosure relates to a method of treating or diminishing a negative symptom of schizophrenia in a patient having a partial but inadequate response to an antipsychotic alone, comprising administering an effective amount of pimavanserin to the patient and continuing administration of the antipsychotic. In certain embodiments, the antipsychotic is selected from the group consisting of quetiapine, clozapine, aripiprazole, asenapine, cariprazine, brexpiprazole, lurasidone, olanzapine, and risperidone. Measurement of a negative symptom of schizophrenia may be performed using a clinical scale such as the negative syndrome subscale of PANSS or the PANSS Marder Negative factor score. In certain embodiments, pimavanserin is administered to the patient in a subchronic dose, e.g., about 5 mg, 10 mg, 17 mg, 20 mg, e.g., about 10 mg to about 20 mg, in combination with administration of antipsychotic.

In some embodiments, pimavanserin may be combined (e.g., administered together in a single dosage form or individually administered with an atypical antipsychotic agent, for example, quetiapine, lurasidone, cariprazine, and asenapine. In some embodiments the antipsychotic may be olanzapine or risperidone. In certain embodiments, a contemplated antipsychotic agent may be clozapine.

In certain embodiments, the effect of pimavanserin may be significant as compared to another 5-HT2A antagonist/inverse agonist, for example, volinanserin (α-(2,3-dimethoxyphenyl)-1-[2-(4-fluorophenylethyl)]-4-piperidinemethanol; M100907), nelotanserin (1-[3-(4-bromo-2-methyl-2H-pyrazol-3-yl)-4-methoxyphenyl]-3-(2,4-difluorophenyl)urea; APD125), esmirtazapine ((S)-1,2,3,4,10,14b-hexahydro-2-methylpyrazino(2,1-a)pyrido(2,3-c)(2)benzazepine), eplivanserin ((E)-1-(2-fluorophenyl)-3-(4-hydroxyphenyl)-2-propen-1-one O-[2-(dimethylamino)ethyl]oxime), and ritanserin (6-[2-[4-[bis(4-fluorophenyl)methylidene]piperidin-1-yl]ethyl]-7-methyl-[1,3]thiazolo[3,2-a]pyrimidin-5-one), lumaLeperone (ITI-007), and roluperidone (2-[[1-[2-(4-fluorophenyl)-2-oxoethyl]piperidin-4-yl]methyl]-3H-isoindol-1-one; MIN-101).

The exact route of administration, dose, or frequency of administration would be readily determined by those skilled in the art and can be dependent on the age, weight, general physical condition, or other clinical symptoms specific to the patient to be treated.

In some embodiments, the tartrate salt of pimavanserin is administered daily. In some embodiments, the tartrate salt of pimavanserin is administered once daily. In some embodiments, the tartrate salt of pimavanserin is formulated for oral administration as a unit dose.

In an embodiment, pimavanserin is administered orally.

In an embodiment, pimavanserin is orally administered in a daily dose from about 0.5 mg to about 90 mg, or from about 8 mg to about 42 mg, or about 10 mg to about 60 mg.

In an embodiment, pimavanserin is orally administered in a daily dose from about 0.5 mg to about 120 mg, or from about 8 mg to about 42 mg, or about 10 mg to about 60 mg.

In certain embodiments, the above ranges are for pimavanserin free base. In certain embodiments, the above ranges are for a pharmaceutically acceptable salt, e.g., a tartrate salt of pimavanserin or any of the salts listed above.

In another embodiment, pimavanserin tartrate is orally administered in a daily dose of about 11.8 mg, 23.6 mg, or 40 mg. In some embodiments, the daily dose is about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 68 mg, about 80 mg, about 88 mg or about 102 mg. In an embodiment the daily dose of pimavanserin tartrate is administered once, twice or three times per day, for example a 40 mg dose of pimavanserin tartrate is administered once a day, or 20 mg pimavanserin tartrate is administered twice a day.

In another embodiment, pimavanserin is orally administered in a daily dose of about 10 mg, 20 mg or 34 mg (mg amounts refer to the amount of pimavanserin free base, which is equivalent to about 11.8 mg, 23.6 mg, or 40 mg pimavanserin tartrate, respectively). In some embodiments, the daily dose is about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 68 mg, about 80 mg, about 88 mg or about 102 mg. In an embodiment the daily dose of pimavanserin is administered once, twice or three times per day, for example a 34 mg dose of pimavanserin is administered once a day, or 17 mg pimavanserin is administered twice a day, or a 10 mg dose pimavanserin is administered once or twice a day for a 10 mg or 20 mg daily dose.

In certain embodiments of the methods provided herein, pimavanserin or a pharmaceutically acceptable salt thereof is orally administered to the subject in a capsule or a tablet, wherein the amount of pimavanserin or pharmaceutically acceptable salt in the capsule or tablet is between 2 mg to 80 mg, between 5 mg to 45 mg, or between 9 mg to 42 mg. For example, the method can comprise administering about 10 mg to about 34 mg daily pimavanserin (equivalent to about 11.8 mg to about 40 mg pimavanserin tartrate). In certain embodiments, the method comprises administering daily about 10 mg, about 20 mg or about 34 mg pimavanserin (equivalent to about 11.8 mg, 23.6 mg, or 40 mg pimavanserin tartrate, respectively).

In certain embodiments, administering an effective amount of pimavanserin comprises: administering about 20 mg of pimavanserin daily for about one to about three weeks; and then administering about 10 mg or about 34 mg pimavanserin daily, e.g., for the duration of treatment. In certain embodiments, administering an effective amount of pimavanserin comprises: administering about 23.6 mg of pimavanserin tartrate daily for about one to about three weeks; and then administering about 11.8 mg or about 40 mg pimavanserin tartrate daily.

Also provided herein is a method of treating loss of normal functions in a patient having a partial but inadequate response to an antipsychotic treatment for schizophrenia, comprising: continuing to administer an effective amount of the antipsychotic; and administering an effective amount of pimavanserin, thereby treating loss of normal functions in the patient suffering from schizophrenia. In some embodiments, the loss of normal function is a negative symptom of schizophrenia.

### EXAMPLES

### Example 1-Pimavanserin as an Adjunctive Therapy Significantly Reduces Negative Symptoms of Schizophrenia

The effect of pimavanserin as an adjunctive treatment in adult schizophrenia patients with a persistent inadequate response (i.e., partial responders) to their existing antipsychotic therapy was tested. 396 patients with moderate-to-severe psychotic symptoms were randomized to receive either pimavanserin or placebo added to existing antipsychotic treatment.

### Summary

Patients adding pimavanserin to existing antipsychotic treatment showed significant improvements on the two measures of negative symptoms, the secondary endpoint PANSS negative symptoms scale sub-score (nominal p=0.047) and the exploratory endpoint PANSS Marder negative factor score (nominal p=0.036).

Further, adding pimavanserin to existing antipsychotic treatment showed a consistent trend in improvement of psychotic symptoms, however the results did not achieve statistical significance on the primary endpoint, the Positive and Negative Syndrome Scale (PANSS) total score (p=0.094). A similar trend was observed on the key secondary endpoint, the Clinical Global Impression-Severity (CGI-S) score (p=0.054). The majority of patients randomized in the study (81.5%) came from European sites and, in this pre-specified subgroup analysis, consistent positive results were observed on both the primary endpoint, PANSS total score (nominal p=0.023), and the key secondary endpoint, CGI-S score (nominal p=0.021).

Pimavanserin was well tolerated with similar rates of adverse events between adjunctive pimavanserin (40.4%) and adjunctive placebo (36.9%). Adverse events reported in at least 5% of patients in the pimavanserin group included headache, somnolence, and insomnia. Additionally, the adjunctive use of pimavanserin did not result in clinically significant differences in vital signs, weight, metabolic syndrome, and extrapyramidal symptoms compared to adjunctive placebo. Approximately 88% of pimavanserin and 96% of placebo patients completed the study. Discontinuations due to adverse event were low for both arms at 2.5% for pimavanserin and 0% for placebo. 1% of patients of subjects in each arm reported serious adverse events.

### Subjects

Subjects enrolled in the study were on a background antipsychotic selected from aripiprazole (oral+Abilify Maintena® & Aristada®), asenapine, cariprazine, brexpiprazole, lurasidone, olanzapine, and risperidone (oral+Risperdal Consta®). Dosages of background oral antipsychotics could not be changed within four weeks of screening and dosages of long-acting injectables (LAI) could not be changed within 16 weeks of screening. Subjects had a history of response to antipsychotic treatment other than clozapine.

There were balanced demographics across treatment groups and overall demographics of 62% male, mean age of 37.2 years old (range between 18 and 55 years), 81.5% European, 87.9% white.

There were balanced baseline disease characteristics across treatment groups, with overall study characteristics as follows:
Mean Duration of Schizophrenia: 11.8 years
Background antipsychotic
   39.1% risperidone (including LAI)
   35.7% olanzapine
   21.3% aripiprazole (including LAI)
Mean PANSS Total Score: 88.2
   Positive Scale Score: 22.9
   Negative Scale Score: 23.0
   General Scale Score: 42.3
Mean CGI-S: 4.6

### Drug Administration

Patients were administered a daily dose of pimavanserin or a placebo for six weeks. The dose started at 20 mg and could be adjusted to 34 mg or 10 mg between weeks 1 and 3. The majority of patients (56%) completed the study at the highest dose level.

### Evaluation

Patients were evaluated at baseline and on a weekly basis according to the clinical rating scale Positive and Negative Symptom Scale (PANSS). The PANSS is a 30-item, 7-point rating system that was developed to cover a range of negative symptoms not adequately measured by the Brief Psychiatric Rating Scale. It has sections that specifically measure positive symptoms, negative symptoms, and general psychopathology in schizophrenic subjects. The PANSS is widely used in trials of antipsychotic drug treatment, and has been formally validated for such use. A subset of 5 of the PANSS factors, the Marder PANSS factors, were also evaluated.

Other clinical rating scales that were assessed were the Clinical Global Impression Scale (CGI), Personal and Social Performance Scale (PSP), drug attitude inventory (DAI-10), Karolinska Sleepiness Scale (KSS), Calgary Depression Scale for Schizophrenia (CDSS), 36-item Short Form Physical Health evaluation (SF36PH) and 36-item Short Form Mental Health evaluation SF36MH.

The Clinical Global Impression Scale (CGI) has: the CGI-Severity (CGI-S), which rates illness severity, and CGI-Improvement (CGI-I), which rates change from the baseline of treatment.

The Personal and Social Performance Scale (PSP) measures a patients' psychosocial functioning in four areas: socially useful activities including work and study, personal and social relationships, self-care, and disturbing and aggressive behavior.

The drug attitude inventory (DAI-10) is a self-reporting measure reflecting a patient's attitude toward psychiatric medication, with a scoring range between −10 and 10. A total score greater than 0 indicates a positive attitude toward psychiatric medications and a total score less than 0 indicates a negative attitude toward psychiatric medications.

The Karolinska Sleepiness Scale (KSS) is a scale for evaluating subjective sleepiness (Kaida et al. Clinical Neurophysiology 2006, 117, 7, 1574-1581). It can be helpful in assessing the effects of drugs. The KSS is a nine point scale (1=extremely alert to 9=extremely sleepy-fighting sleep). “Karolinska Sleepiness Scale (KSS)”

The Calgary Depression Scale for Schizophrenia (CDSS) is a 9-item, 4-point scale that was specifically designed to measure depressive symptoms in schizophrenic subjects, separate from the positive, negative, and extrapyramidal symptoms observed in this population. It has been widely used in treatment trials in schizophrenia and has been validated for such use.

### Results

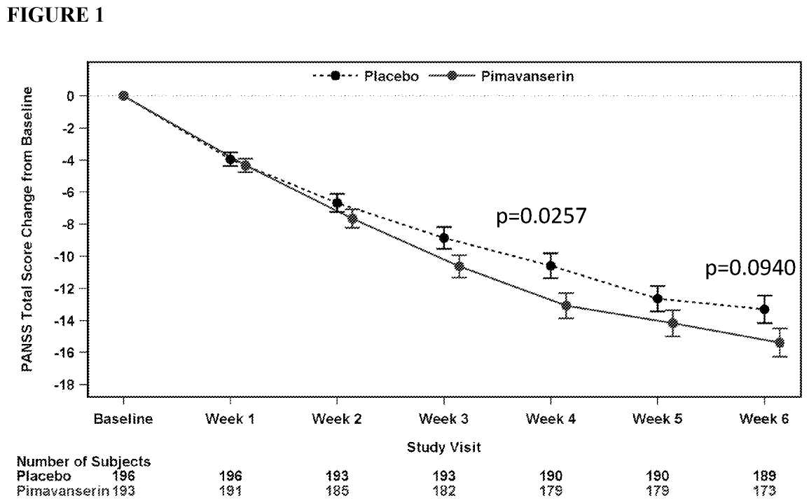

The primary endpoint of the study was a change in the PANSS total score after 6 weeks of treatment. As shown in FIG. 1, adding pimavanserin to existing antipsychotic treatment showed a consistent trend in improvement of psychotic symptoms. However the results did not achieve statistical significance on the primary endpoint, the Positive and Negative Syndrome Scale (PANSS) total score (p=0.094) (see FIG. 1 and TABLE 1).

TABLE 1

| | | |
|---|---|---|
| Full Analysis Set | Placebo | Pimavanserin |

Baseline

| | | |
|---|---|---|
| n | 196 | 193 |
| Mean (SE) | 88.1 (0.61) | 88.3 (0.68) |

Week 6

| | | |
|---|---|---|
| N | 189 | 173 |
| Mean (SE) | 74.6 (1.01) | 72.7 (1.04) |

Change from Baseline to Week 6

| | | |
|---|---|---|
| N | 189 | 173 |
| Mean (SE) | −13.4 (0.83) | −15.3 (0.93) |
| MMRM LSM (SE) [1] | −13.3 (0.86) | −15.4 (0.89) |
| 95% CI | (−15.0, −11.6) | (−17.1, −13.6) |
| Diff in MMRM LSM (SE) [2] | | −2.1 (1.24) |
| 95% CI of Diff | | (−4.5, 0.4) |
| MMRM p-value [3] | | 0.0940 |
| Effect Size (Cohen’s d) | | 0.173 |

[1] LSM from MMRM with fixed effects of region (North America, Europe, rest of world), planned treatment (adjunctive pimavanserin, adjunctive placebo), study visit (Weeks 1, 2, 3, 4, 5, 6), treatment-by-visit interaction, Baseline score (continuous covariate), and Baseline-by-visit interaction. An unstructured covariance matrix is used to model the within-subject errors. The denominator degrees of freedom are estimated using the Kenward-Roger approximation.
[2] Difference between LSM changes for adjunctive pimavanserin and adjunctive placebo (pimavanserin − placebo) at the specified visit from MMRM analysis.
[3] 2-sided p-value for treatment difference at specified visit from MMRM analysis.

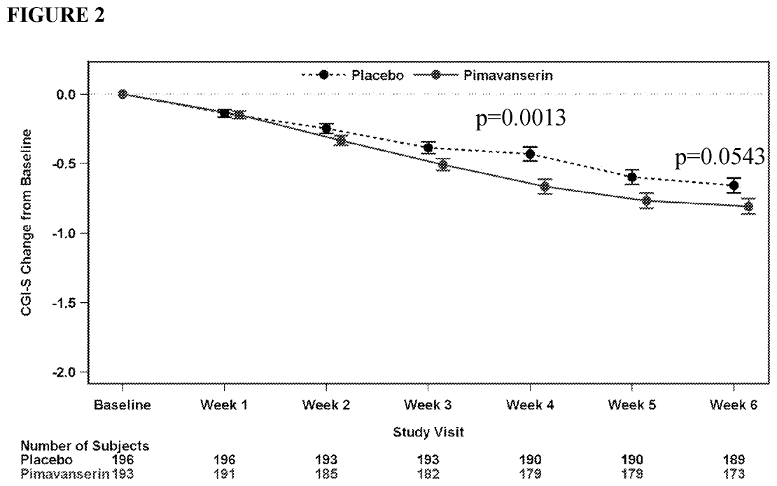

As shown in FIG. 2 and TABLE 2, a similar trend was observed on the key secondary endpoint, the Clinical Global Impression-Severity (CGI-S) score (p=0.054).

TABLE 2

| | | |
|---|---|---|
| Full Analysis Set | Placebo | Pimavanserin |

Baseline

| | | |
|---|---|---|
| n | 196 | 193 |
| Mean (SE) | 4.6 (0.04) | 4.6 (0.04) |

Week 6

| | | |
|---|---|---|
| N | 189 | 173 |
| Mean (SE) | 3.9 (0.06) | 3.8 (0.07) |

Change from Baseline to Week 6

| | | |
|---|---|---|
| N | 189 | 173 |
| Mean (SE) | −0.7 (0.05) | −0.8 (0.06) |
| MMRM LSM (SE) [1] | −0.7 (0.05) | −0.8 (0.06) |
| 95% CI | (−0.8, −0.6) | (−0.9, −0.7) |
| Diff in MMRM LSM (SE) [2] | | −0.1 (0.08) |
| 95% CI of Diff | | (−0.3, 0.0) |
| MMRM p-value [3] | | 0.0543 |
| Effect Size (Cohen’s d) | | 0.200 |

[1] LSM from MMRM with fixed effects of region (North America, Europe, rest of world), planned treatment (adjunctive pimavanserin, adjunctive placebo), study visit (Weeks 1, 2, 3, 4, 5, 6), treatment-by-visit interaction, Baseline score (continuous covariate), and Baseline-by-visit interaction. An unstructured covariance matrix is used to model the within-subject errors. The denominator degrees of freedom are estimated using the Kenward-Roger approximation.
[2] Difference between LSM changes for adjunctive pimavanserin and adjunctive placebo (pimavanserin − placebo) at the specified visit from MMRM analysis.

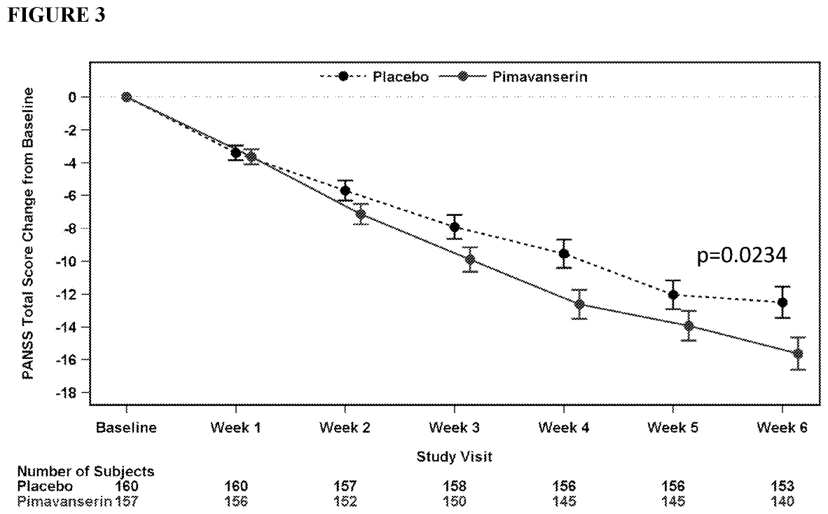

Although statistical significance was not achieved across the study as a whole, the majority of patients randomized in the study (81.5%) came from European sites and, in this pre-specified subgroup analysis, consistent positive results were observed on both the primary endpoint, PANSS total score (nominal p=0.023; see TABLE 3 and FIG. 3, compare to PANSS total score for North American sites p=0.3402, data not shown), and the key secondary endpoint, CGI-S score (nominal p=0.021; TABLE 4; compare to CGI-S score for North American sites p=0.5529, data not shown).

TABLE 3

| | | |
|---|---|---|
| Full Analysis Set | Placebo | Pimavanserin |

Baseline

| | | |
|---|---|---|
| n | 160 | 157 |
| Mean (SE) | 88.8 (0.65) | 89.2 (0.72) |

Week 6

| | | |
|---|---|---|
| N | 153 | 140 |
| Mean (SE) | 76.2 (1.06) | 73.2 (1.18) |

Change from Baseline to Week 6

| | | |
|---|---|---|
| N | 153 | 140 |
| Mean (SE) | −12.6 (0.93) | −16.0 (1.06) |
| MMRM LSM (SE) [1] | −12.5 (0.96) | −15.6 (0.98) |
| 95% CI | (−14.4, −10.6) | (−17.6, −13.7) |
| Diff in MMRM LSM (SE) [2] | | −3.1 (1.37) |
| 95% CI of Diff | | (−5.8, −0.4) |
| MMRM p-value [3] | | 0.0234 |
| Effect Size (Cohen’s d) | | 0.261 |

[1] LSM from MMRM with fixed effects of region (North America, Europe, rest of world), planned treatment (adjunctive pimavanserin, adjunctive placebo), study visit (Weeks 1, 2, 3, 4, 5, 6), treatment-by-visit interaction, Baseline score (continuous covariate), and Baseline-by-visit interaction. An unstructured covariance matrix is used to model the within-subject errors. The denominator degrees of freedom are estimated using the Kenward-Roger approximation.
[2] Difference between LSM changes for adjunctive pimavanserin and adjunctive placebo (pimavanserin − placebo) at the specified visit from MMRM analysis.
[3] 2-sided p-value for treatment difference at specified visit from MMRM analysis.

TABLE 4

| | | |
|---|---|---|
| Full Analysis Set | Placebo | Pimavanserin |

Baseline

| | | |
|---|---|---|
| n | 160 | 157 |
| Mean (SE) | 4.6 (0.04) | 4.6 (0.04) |

Week 6

| | | |
|---|---|---|
| N | 153 | 140 |
| Mean (SE) | 4.0 (0.06) | 3.8 (0.08) |

Change from Baseline to Week 6

| | | |
|---|---|---|
| N | 153 | 140 |
| Mean (SE) | −0.6 (0.06) | −0.8 (0.07) |
| MMRM LSM (SE) [1] | −0.6 (0.06) | −0.8 (0.06) |
| 95% CI | (−0.7, −0.5) | (−1.0, −0.7) |
| Diff in MMRM LSM (SE) [2] | | −0.2 (0.09) |
| 95% CI of Diff | | (−0.4, −0.0) |
| MMRM p-value [3] | | 0.0214 |
| Effect Size (Cohen’s d) | | 0.266 |

[1] LSM from MMRM with fixed effects of region (North America, Europe, rest of world), planned treatment (adjunctive pimavanserin, adjunctive placebo), study visit (Weeks 1, 2, 3, 4, 5, 6), treatment-by-visit interaction, Baseline score (continuous covariate), and Baseline-by-visit interaction. An unstructured covariance matrix is used to model the within-subject errors. The denominator degrees of freedom are estimated using the Kenward-Roger approximation.
[2] Difference between LSM changes for adjunctive pimavanserin and adjunctive placebo (pimavanserin − placebo) at the specified visit from MMRM analysis.
[3] 2-sided p-value for treatment difference at specified visit from MMRM analysis.

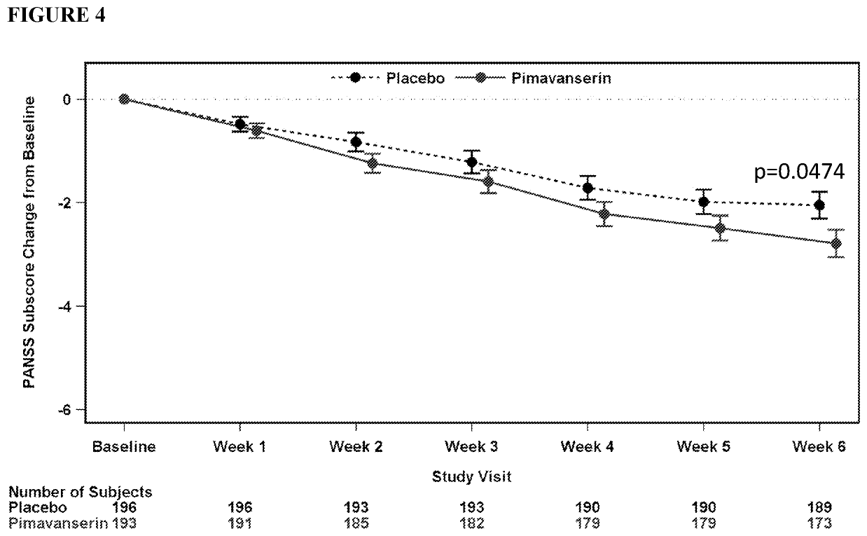

Patients adding pimavanserin to existing antipsychotic treatment showed significant improvements on the two measures of negative symptoms, the secondary endpoint PANSS negative symptoms scale sub-score (nominal p=0.047; FIG. 4 and TABLE 5) and the exploratory endpoint PANSS Marder negative factor score (nominal p=0.036; TABLE 6), indicating that the addition of pimavanserin to an existing antipsychotic treatment can diminish negative symptoms of schizophrenia.

TABLE 5

| | | |
|---|---|---|
| Full Analysis Set | Placebo | Pimavanserin |

Baseline

| | | |
|---|---|---|
| n | 196 | 193 |
| Mean (SE) | 23.1 (0.29) | 23.0 (0.29) |

Week 6

| | | |
|---|---|---|
| N | 189 | 173 |
| Mean (SE) | 20.9 (0.34) | 20.3 (0.35) |

Change from Baseline to Week 6

| | | |
|---|---|---|
| N | 189 | 173 |
| Mean (SE) | −2.1 (0.28) | −2.8 (0.28) |
| MMRM LSM (SE) [1] | −2.0 (0.26) | −2.8 (0.27) |
| 95% CI | (−2.6, −1.5) | (−3.3, −2.3) |
| Diff in MMRM LSM (SE) [2] | | −0.7 (0.37) |
| 95% CI of Diff | | (−1.5, −0.0) |
| MMRM p-value [3] | | 0.0474 |
| Effect Size (Cohen’s d) | | 0.206 |

[1] LSM from MMRM with fixed effects of region (North America, Europe, rest of world), planned treatment (adjunctive pimavanserin, adjunctive placebo), study visit (Weeks 1, 2, 3, 4, 5, 6), treatment-by-visit interaction, Baseline score (continuous covariate), and Baseline-by-visit interaction. An unstructured covariance matrix is used to model the within-subject errors. The denominator degrees of freedom are estimated using the Kenward-Roger approximation.
[2] Difference between LSM changes for adjunctive pimavanserin and adjunctive placebo (pimavanserin − placebo) at the specified visit from MMRM analysis.
[3] 2-sided p-value for treatment difference at specified visit from MMRM analysis.

TABLE 6

| | | |
|---|---|---|
| Full Analysis Set | Placebo | Pimavanserin |

Baseline

| | | |
|---|---|---|
| n | 196 | 193 |
| Mean (SE) | 22.5 (0.30) | 22.3 (0.33) |

Week 6

| | | |
|---|---|---|
| N | 189 | 173 |
| Mean (SE) | 20.0 (0.37) | 19.2 (0.38) |

Change from Baseline to Week 6

| | | |
|---|---|---|
| N | 189 | 173 |
| Mean (SE) | −2.6 (0.31) | −3.3 (0.31) |
| MMRM LSM (SE) [1] | −2.5 (0.29) | −3.4 (0.30) |
| 95% CI | (−3.1, −1.9) | (−4.0, −2.8) |
| Diff in MMRM LSM (SE) [2] | | −0.9 (0.41) |
| 95% CI of Diff | | (−1.7, −0.1) |
| MMRM p-value [3] | | 0.0362 |
| Effect Size (Cohen’s d) | | 0.218 |

[1] LSM from MMRM with fixed effects of region (North America, Europe, rest of world), planned treatment (adjunctive pimavanserin, adjunctive placebo), study visit (Weeks 1, 2, 3, 4, 5, 6), treatment-by-visit interaction, Baseline score (continuous covariate), and Baseline-by-visit interaction. An unstructured covariance matrix is used to model the within-subject errors. The denominator degrees of freedom are estimated using the Kenward-Roger approximation.
[2] Difference between LSM changes for adjunctive pimavanserin and adjunctive placebo (pimavanserin − placebo) at the specified visit from MMRM analysis.
[3] 2-sided p-value for treatment difference at specified visit from MMRM analysis.

Individual item scores showing improvement at 4 weeks with pimavanserin included hallucinatory behavior (p=0.0111), hostility (p=0.0096), passive/apathetic social withdrawal (p=0.0099), poor impulse control (p=0.0862). Individual item scores showing improvement at 6 weeks with pimavanserin included blunted effect (p=0.0142).

A summary of the efficacy endpoints is shown in TABLE 7. As shown, adding pimavanserin to an existing antipsychotic treatment is also effective in treating an aspect of sleep disturbance associated with schizophrenia. Specifically, patients taking pimavanserin showed an improved score on the Karolinska Sleepiness Scale, as compared to patients taking placebo (p=0.0265).

TABLE 7
FAS Population, OC

| | | | | LSM (SE) | | | | | | |
|---|---|---|---|---|---|---|---|---|---|---|
| | | Mean (SD) at | | Change from | | Treatment group Comparison | | | | |
| | | Baseline | | Baseline at W6 | | (PIM-PBO) | | | | |
| | | PBO | PIM | PBO | PIM | | LSM | 95% | | Effect |
| Numeric Endpoints | | N = 196* | N = 193* | N = 189* | N = 173* | Model | (SE) | CI | P-val | Size |
| Primary | PANSS | 88.1 | 88.3 | −13.3 | −15.4 | MMRM | −2.1 | (−4.5, | 0.0940 | 0.17 |
| | Total | (8.58) | (9.40) | (0.86) | (0.89) | | (1.24) | 0.4) | | |
| Sensitivity | OC in PP | 88.1 | 88.4 | −13.3 | −15.6 | MMRM | −2.3 | (−4.7, | 0.0646 | 0.19 |
| | | (8.53) | (9.24) | (0.86) | (0.88) | | (1.23) | 0.1) | | |
| | MI in | 88.1 | 88.3 | −13.4 | −15.2 | ANCOVA | −1.9 | (−4.3, | 0.1316 | 0.15 |
| | RAND | (8.58) | (9.34) | (0.86) | (0.88) | | (1.23) | 0.6) | | |
| | MI in | 88.1 | 88.3 | −13.4 | −15.3 | ANCOVA | −1.9 | (−4.3, | 0.1203 | 0.16 |
| | FAS | (8.58) | (9.40) | (0.86) | (0.88) | | (1.23) | 0.5) | | |
| | OC | 88.1 | 88.3 | −13.4 | −15.3 | ANCOVA | −1.9 | (−4.4, | 0.1183 | 0.16 |
| | ANCOVA | (8.58) | (9.40) | (0.85) | (0.89) | | (1.24) | 0.6) | | |
| | Non- | | | | | Van | | | 0.8241 | |
| | Parametric | | | | | Elteren | | | | |
| Key 2^{nd} | CGI-S | 4.6 | 4.6 | −0.7 | −0.8 | MMRM | −0.1 | (−0.3, | 0.0543 | 0.20 |
| | | (0.52) | (0.55) | (0.05) | (0.06) | | (0.08) | 0.0) | | |
| Secondary | CGI-I | — | — | 2.9 | 2.9 | MMRM | −0.1 | (−0.3, | 0.4205 | 0.08 |
| | | | | (0.07) | (0.07) | | (0.10) | 0.1) | | |
| | PANSS | 22.8 | 23.0 | −4.9 | −5.4 | MMRM | −0.5 | (−1.4, | 0.2467 | 0.12 |
| | Pos | (3.21) | (3.44) | (0.30) | (0.31) | | (0.43) | 0.4) | | |
| | PANSS | 23.1 | 23.0 | −2.0 | −2.8 | MMRM | −0.7 | (−1.5, | 0.0474 | 0.21 |
| | Neg | (4.00) | (4.09) | (0.26) | (0.27) | | (0.37) | −0.0) | | |
| | PANSS | 42.2 | 42.4 | −6.4 | −7.2 | MMRM | −0.8 | (−2.1, | 0.2156 | 0.12 |
| | GenPsy | (5.93) | (6.21) | (0.46) | (0.47) | | (0.66) | 0.5) | | |
| | PSP | 51.6 | 51.8 | 5.6 | 6.9 | ANCOVA | 1.3 | (−0.5, | 0.1598 | 0.15 |
| | | (10.91) | (10.95) | (0.62) | (0.64) | | (0.89) | 3.0) | | |
| | DAI-10 | 5.8 | 5.6 | 0.4 | 0.4 | ANCOVA | −0.0 | (−0.5, | 0.8879 | Neg |
| | | (3.27) | (3.32) | (0.18) | (0.18) | | (0.25) | 0.5) | | |
| | KSS | 4.7 | 4.6 | −0.2 | −0.5 | MMRM | −0.3 | (−0.6, | 0.0265 | 0.23 |
| | | (1.65) | (1.52) | (0.10) | (0.10) | | (0.14) | −0.0) | | |
| Exploratory | Marder | 27.7 | 27.9 | −4.7 | −5.5 | MMRM | −0.8 | (−1.7, | 0.1164 | 0.16 |
| | Pos | (3.39) | (3.89) | (0.33) | (0.34) | | (0.48) | 0.2) | | |
| | Marder | 22.5 | 22.3 | −2.5 | −3.4 | MMRM | −0.9 | (−1.7, | 0.0362 | 0.22 |
| | Neg | (4.19) | (4.52) | (0.29) | (0.30) | | (0.41) | −0.1) | | |
| | Marder | 19.2 | 19.7 | −2.0 | −2.5 | MMRM | −0.5 | (−1.1, | 0.0904 | 0.18 |
| | Disorg | (3.39) | (3.88) | (0.21) | (0.22) | | (0.31) | 0.1) | | |
| | Marder | 8.5 | 8.4 | −1.7 | −1.7 | MMRM | −0.0 | (−0.5, | 0.9419 | 0.01 |
| | Uncntl | (3.12) | (3.08) | (0.16) | (0.17) | | (0.24) | 0.4) | | |
| | Marder | 10.2 | 10.0 | −2.3 | −2.3 | MMRM | −0.0 | (−0.6, | 0.8788 | 0.02 |
| | AnxDep | (3.02) | (2.98) | (0.19) | (0.20) | | (0.28) | 0.5) | | |
| | CDSS | 2.1 | 1.9 | −0.4 | −0.6 | ANCOVA | −0.2 | (−0.5, | 0.3726 | 0.09 |
| | | (2.64) | (2.08) | (0.12) | (0.12) | | (0.17) | 0.2) | | |
| | SF36 PH | 52.8 | 52.7 | 0.44 | 0.46 | ANCOVA | 0.0 | (−1.32, | 0.9843 | 0.00 |
| | | (8.57) | (8.13) | (0.47) | (0.49) | | (0.68) | 1.34) | | |
| | SF36 MH | 36.7 | 38.0 | 4.6 | 5.6 | ANCOVA | 1.0 | (−0.97, | 0.3210 | 0.11 |
| | | (11.06) | (11.29) | (0.69) | (0.72) | | (1.00) | 2.95) | | |

FAS population

| | | W6 | | W6 | | Treatment Group | | |
|---|---|---|---|---|---|---|---|---|
| | | PBO | | PIM | | Comparison (PIM − PBO) | | |
| Categorical Endpoints | | N | n (%) | N | n (%) | Diff in % | 95% CI | p-val |
| Missing as | PANSS >= 20% | 196 | 99 | 193 | 109 | 5.9 | (−4.0, 15.6) | 0.2396 |
| non-Rsp | Rsp | | (50.5) | | (56.5) | | | |
| | PANS S >= 30% | 196 | 67 | 193 | 71 | 2.6 | (−6.9, 12.0) | 0.5955 |
| | Rsp | | (34.2) | | (36.8) | | | |
| | CGI-I 1 or 2 Rsp | 196 | 65 | 193 | 68 | 2.1 | (−7.3, 11.4) | 0.6681 |
| | | | (33.2) | | (35.2) | | | |
| Missing as | CGI-I no Chg or | 196 | 60 | 193 | 67 | 4.1 | (−5.2, 13.3) | 0.3859 |
| Worsen | Worsened | | (30.6) | | (34.7) | | | |
| OC | PANSS >= 20% | 189 | 99 | 173 | 109 | 10.6 | (0.4, 20.5) | 0.0406 |
| | Rsp | | (52.4) | | (63.0) | | | |
| | PANSS >= 30% | 189 | 67 | 173 | 71 | 5.6 | (−4.4, 15.5) | 0.2739 |
| | Rsp | | (35.4) | | (41.0) | | | |
| | CGI-I 1 or 2 Rsp | 189 | 65 | 173 | 68 | 4.9 | (−5.0, 14.7) | 0.3339 |
| | | | (34.4) | | (39.3) | | | |
| | CGI-I no Chg or | 189 | 53 | 173 | 47 | −0.9 | (−10.0, 8.4) | 0.8530 |
| | Worsened | | (28.0) | | (27.2) | | | |

*Counts based on #pts with non-missing OC PANSS total score in FAS just for reference purpose. Actual counts for other endpoints and/or analysis sets vary.

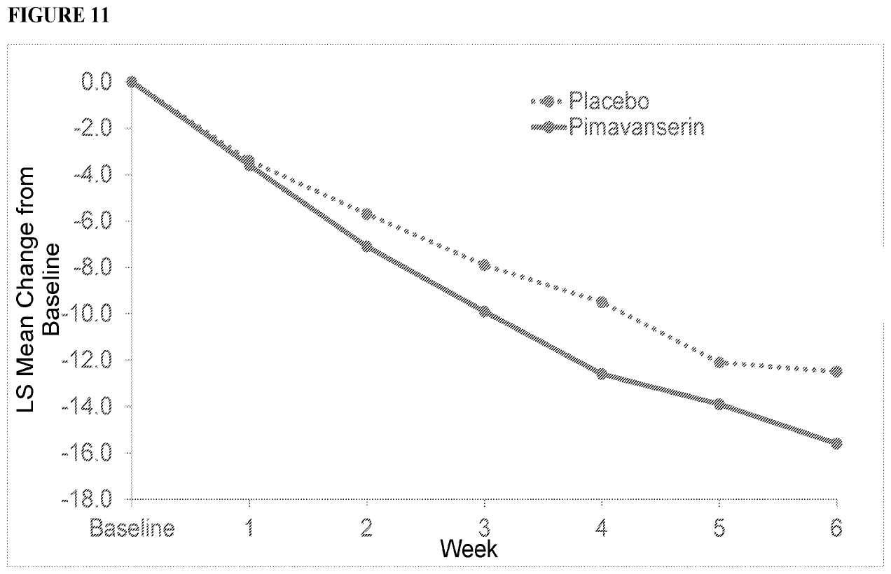

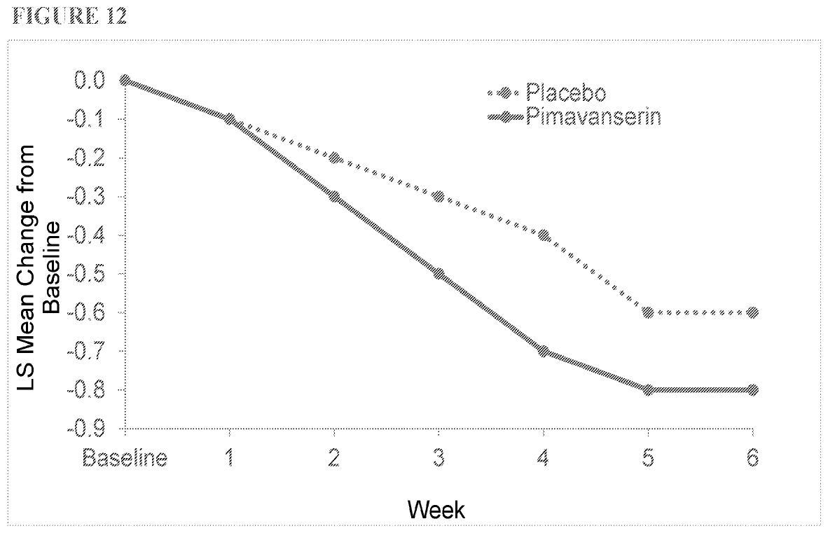

The majority of patients (81.5%) were enrolled in European sites. A pre-specified analysis by region (Europe and North America) revealed a significant treatment effect for pimavanserin vs. placebo on both the PANSS Total Score (LSmean difference: −3.1, 95% CI: −5.8, −0.4, unadjusted p=0.023) and the CGI-S score (LSmean difference: −0.2, 95% CI: −0.4, 0, unadjusted p=0.021) in the European region (FIGS. 11 and 12 and Table 8). Significant improvement for pimavanserin vs. placebo on the CGI-S also were observed at Weeks 3 (unadjusted p=0.034), 4 (unadjusted p=0.0009, and 5 (unadjusted p=0.018) for the European region (FIG. 12).

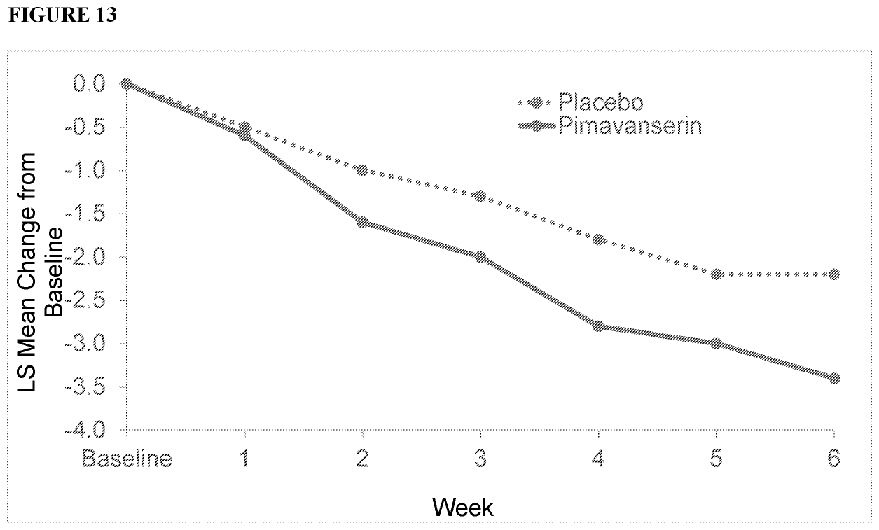

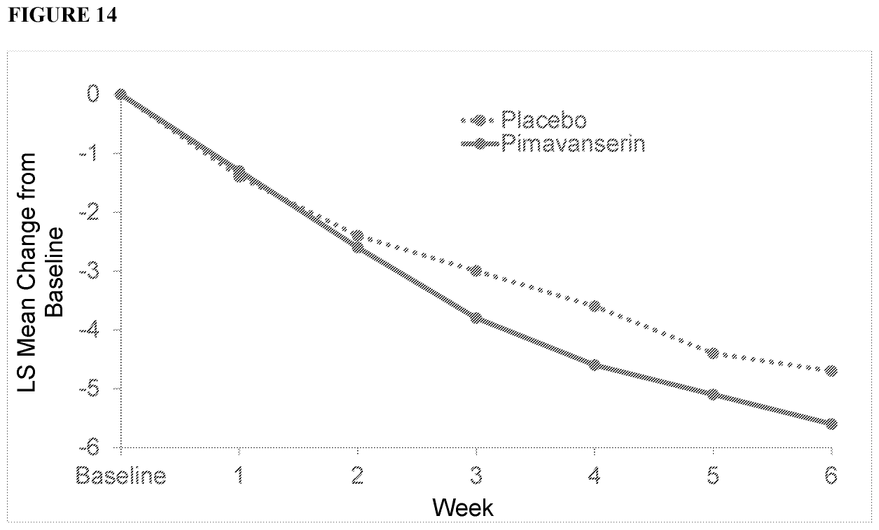

For the PANSS Marder Factor score in the FAS, a significant difference favoring pimavanserin vs. placebo was observed at Week 6 (unadjusted p=0.036) for negative symptoms (Table 7). In European sites, differences favored pimavanserin vs. placebo at Week 3 (unadjusted p=0.027) and Week 4 (unadjusted p=0.017) for PANSS Marder Positive Symptoms Factor Score and at Weeks 2, 3, 4, and 6 for the PANSS Marder Negative Symptom Factor Score (unadjusted p=0.004 at Week 6) (FIGS. 13 and 14). For the PANSS Negative Symptoms subscale, a significant (unadjusted p=0.011) difference in favor of pimavanserin vs. placebo was observed at Week 6 in the European region. No significant differences between treatment groups were observed for either positive or negative symptoms for North America.

TABLE 8

| | Placebo (N = 196) | | Pimavanserin (N = 193) | | | | |
|---|---|---|---|---|---|---|---|
| | | LS | | LS | | | |
| | | Mean | | Mean | | | |
| | | (SE) | | (SE) | | | |
| | | Change | | Change | LS | | |
| | | at | | at | Mean (SE) | | |
| | Mean (SE) | Week | Mean (SE) | Week | Difference | | Cohen's d |
| Endpoint | Baseline | 6 | Baseline | 6 | 95% CI | p-value | Effect Size |
| PANSS | 88.8 (0.65) | −12.5 | 89.2 (0.72) | −15.6 | −3.1 (1.37) | 0.023 | 0.261 |
| Total Score— | n = 160 | (0.96) | n = 157 | (0.98) | [−5.8, −0.4] | | |
| Europe | | | | | | | |
| PANSS | 84.7 (1.59) | −16.8 | 84.7 (1.71) | −14.2 | 2.6 (2.74) | 0.340 | −0.229 |
| Total Score— | n = 36 | (1.91) | n = 36 | (1.96) | [−2.8, 8.1] | | |
| North | | | | | | | |
| America | | | | | | | |
| Clinical | 4.6 (0.04) | −0.6 | 4.6 (0.04) | −0.8 | −0.2 (0.09) | 0.021 | 0.266 |
| Global | n = 160 | (0.06) | n = 157 | (0.06) | [−0.4, 0.0] | | |
| Impressions- | | | | | | | |
| Severity— | | | | | | | |
| Europe | | | | | | | |
| Clinical | 4.4 (0.08) | −0.8 | 4.6 (0.09) | −0.7 | 0.1 (0.15) | 0.553 | −0.144 |
| Global | n = 36 | (0.1) | n = 36 | (0.11) | 0.2 [−0.2, 0.4] | | |
| Impressions- | | | | | | | |
| Severity— | | | | | | | |
| North | | | | | | | |
| America | | | | | | | |
| PANSS | 22.7 (0.24 | −4.7 | 23.0 (0.28) | −5.5 | −0.8 (0.49) | 0.089 | 0.195 |
| Positive | n = 160 | (0.34) | n = 157 | (0.35) | [−1.8, 0.1] | | |
| Symptoms | | | | | | | |
| Subscale— | | | | | | | |
| Europe | | | | | | | |
| PANSS | 23.5 (0.31) | −1.9 | 23.6 (0.29) | −2.9 | −1.1 (0.41 | 0.011 | 0.296 |
| Negative | n = 160 | (0.29) | n = 157 | (0.29) | [−1.9, −0.2] | | |
| Symptoms | | | | | | | |
| Subscale— | | | | | | | |
| Europe | | | | | | | |

### Safety

The safety of the pimavanserin arm was similar to placebo, with no new signals observed. As shown in TABLE 9, the overall adverse effect (AE) profile was similar to placebo with slightly higher incidence of AEs in active arm. The overall number of SAEs and AEs leading to discontinuation was low. The most commonly reported AEs leading to discontinuation of treatment were from the psychiatric disorders system and seen in the active arm. No effect was seen on vital signs, weight, metabolic syndrome, and extrapyramidal symptoms. Minimal QT prolongation was observed.

TABLE 9

| | Placebo | | Pimavanserin | |
|---|---|---|---|---|
| | (N = 198) | | (N = 198) | |
| | Subjects | Events | Subjects | Events |
| Safety Analysis Set | n (%) | n | n (%) | n |
| Any Treatment-Emergent | 73 (36.9) | 141 | 80 (40.4) | 142 |
| Adverse Event | | | | |
| Any Serious Treatment- | 2 (1.0) | 3 | 2 (1.0) | 3 |
| Emergent Adverse Event | | | | |
| Any Related[1] Treatment- | 25 (12.6) | 46 | 33 (16.7) | 46 |
| Emergent Adverse Event | | | | |
| Any Related[1] Serious | | | 1 (0.5) | 2 |
| Treatment-Emergent Adverse | | | | |
| Event | | | | |
| Any Treatment-Emergent | | | 5 (2.5) | 7 |
| Adverse Event Leading to | | | | |
| Discontinuation or Study | | | | |
| Termination | | | | |
| Any Treatment-Emergent | | | | |
| Adverse Event Resulting in Death | | | | |

### Example 2—Effects of Atypical Antipsychotic Drugs and Pimavanserin on Social Interaction in a Mouse Model of Treatment-Resistant Negative Symptoms of Schizophrenia

Experiments were conducted to establish an animal model of treatment resistance in negative symptoms of schizophrenia, and then test the efficacy of pimavanserin, atypical antipsychotics, or combinations of pimavanserin with atypical antipsychotics (risperidone, olanzapine and aripiprazole) against negative symptoms. Social interaction (SI) was used as a surrogate measure in mice for negative symptoms in humans, and treatment resistance was achieved by combining subchronic-phencyclidine (scPCP) treatment with acute restraint stress (ARS).

### Methods

Negative symptoms model: The behavioral assay utilized to assess negative symptoms was social interaction (SI) task.

Briefly, mice were habituated to the test arena (i.e., the NOR box) for 30 minutes prior to the test day. Pairs of mice, unfamiliar to each other, received either the same treatment (e.g., vehicle and vehicle; N=10 pairs) or different treatments (e.g., vehicle; N=10 and phencyclidine (PCP) 10 mg/kg; N=10). After washout, they were placed in the test arena together for 10 minutes. A heavy object made of Plexiglas or metal was placed in the center of the test arena to quantify the time and nature of interaction of the test animals with an unfamiliar object or an unfamiliar animal. After the 10 min test phase, the object and the arena were cleaned with 70% ethanol to remove any olfactory trails. Behavior was recorded on video for subsequent blind scoring.

The following four behaviors were scored for each animal:
a) Sniffing: A normal mouse engages in sniffing the con-specific mouse's body, including the anogenital area
b) Following: A normal mouse pursues a con-specific mouse around the arena
c) Avoiding: A normal mouse avoids the con-specific mouse and move away from a pursuing mouse if approached
d) Object Exploration: A normal mouse explores the object that is placed in the center of the test arena.

Acute restraint stress (ARS) protocol: Mice were assigned to one of two groups: either the control group (non-stress) or the stress group. Generally, mice of the stress group were restrained in well-ventilated 50 mL tubes. After the restraint period of 1 or 2 hours, the mice were transferred into a clean animal housing cage for 1-hour interval and thereafter went through the first social interaction test (different mice are used to test different groups).

Treatment resistant animal model: To establish this model, Acute restraint stress (ARS) was added to scPCP treatment. Two groups (vehicle+ARS; scPCP+ARS) and their controls (vehicle+saline; scPCP+saline) were used. ARS was applied for 1 or 2 hours and tested on SI 1 hour post-ARS.

scPCP treatment involved intraperitoneal injection (ip) of 10 mg/kg PCP to male C57BL/6J mice, twice daily (bid) for 7 days, followed by 7 days washout. To test the effect of atypical APD or pimavanserin in scPCP-treated mice (N=10 pairs of mice per group), risperidone was administered as 0.05 mg/kg ip or 0.2 mg/kg ip; and pimavanserin was administered as 3 mg/kg ip or 6 mg/kg ip to scPCP-treated mice 45 minutes prior to the commencement of the social interaction task in the 10 min social interaction test.

### Results

Subchronic treatment with phencyclidine (PCP, an NMDA receptor antagonist) in mice produced social interaction (SI) deficits which were increased when the mice were subjected to ARS in addition to receiving scPCP. scPCP treatment produced a profound deficit in sniffing, no deficit in following, and a mild deficit in avoiding in the mice model. Mice receiving scPCP and ARS showed further decreases in following, and further increases in avoiding and object exploration. These data establishes that scPCP produces a model of negative symptoms in mice that is further enhanced by addition of ARS.

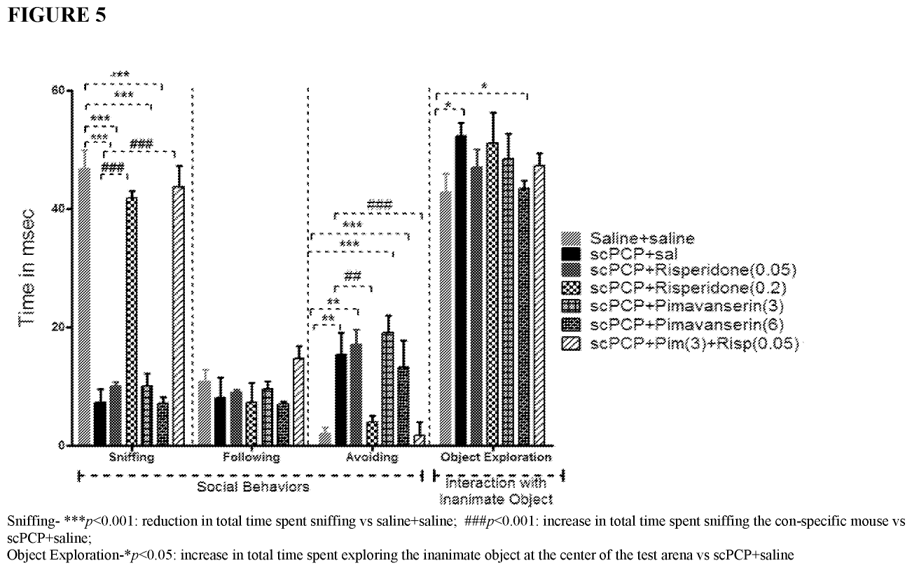

As shown in FIG. 5, scPCP-treated mice (test mice) given risperidone (0.2 mg/kg, but not 0.05 mg/kg) spent more time exploring the conspecific mouse, similar to the control mouse, thus establishing efficacy for risperidone to overcome the SI deficit produced by scPCP alone. Pimavanserin (3 and 6 mg/kg) alone did not restore SI deficit in scPCP-treated mice. However the combination of sub-effective (SED) dose of risperidone (0.05 mg/kg) and pimavanserin (3 mg/kg) rescued the SI deficit produced by scPCP alone. These results show that an APD alone can restore SI in mice with SI deficit due to scPCP, and also has value as a model of non-treatment resistant SI deficit. This shows that pimavanserin can augment SED risperidone, indicating that 5-HT2A antagonism is a relevant treatment for this model of negative symptoms.

### Example 3—Combination of Pimavanserin and Atypical APD Rescues Social Interaction Deficit in scPCP-Treated Mice Given 2 Hours of Acute Restraint Stress

The effect of acute atypical APDs (risperidone, olanzapine, and aripiprazole) and 5-HT2AR inverse agonist pimavanserin, alone or in combination, on social interaction was tested in scPCP-treated mice given 2 h of ARS.

### Methods

The behavioral assay, ARS protocol, and scPCP-treatment as described in Example 2 were used. To test the effect of atypical APD and pimavanserin, alone or as con-administration, in scPCP-treated mice (N=10 pairs of mice per group) given 2 h ARS, risperidone (“Risp”) was administered as 0.2 mg/kg or 0.4 mg/kg ip; olanzapine (“Olan”) as 1.0 mg/kg ip; aripiprazole (“Aripi”) as 1.0 mg/kg ip; and pimavanserin (“Pim”) as 3 mg/kg ip to scPCP+ARS(2h) treated male C57BL/6J mice 45 minutes prior to the commencement of the social interaction task in the 10 min social interaction test. Plasma levels of risperidone were measured using a Triple Quad 6500+ mass spectrometer.

### Results

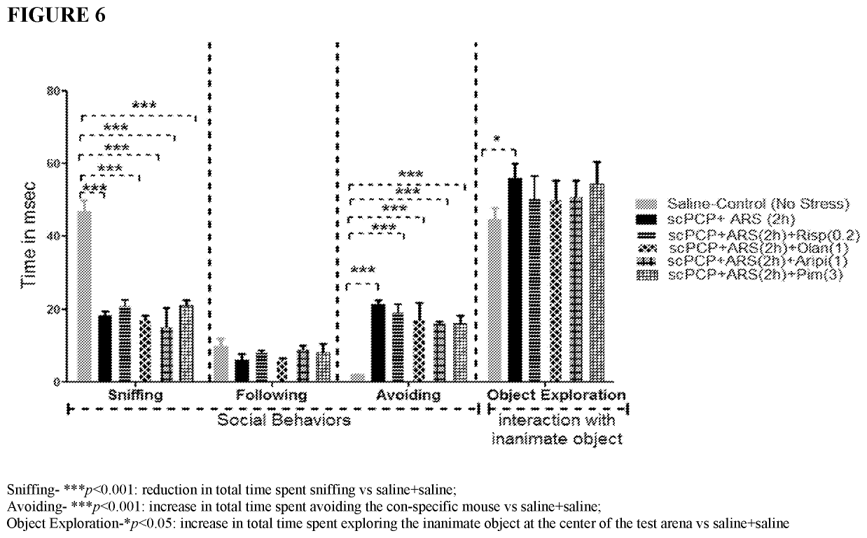

As shown in FIG. 6, the scPCP mouse (test mouse) given a single 2 h episode of restraint showed a significant deficit in SI as indicated by the significantly decreased sniffing time, and increased avoidance. Neither risperidone (0.2 mg/kg), olanzapine (1 mg/kg), aripiprazole (1 mg/kg), or pimavanserin (3 mg/kg) alone were able to restore sniffing or decrease avoidance. Risperidone, which rescued the scPCP-induced SI deficit, did not rescue SI deficit in scPCP-treated mice subjected to 2h ARS.

This establishes scPCP+ARS (2h) as a model for treatment-resistant negative symptoms based on the definition of treatment resistance (TR) being failure to respond to adequate doses of two or more APDs.

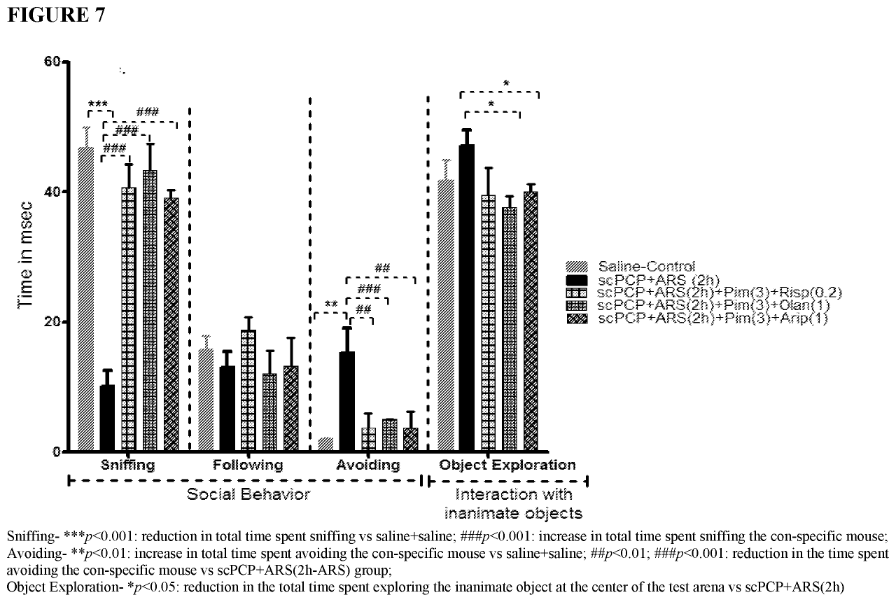

In contrast, as shown in FIG. 7, the combination of pimavanserin (3 mg/kg)+risperidone (0.2 mg/kg), pimavanserin (3 mg/kg)+olanzapine (1 m/kg), or pimavanserin (3 mg/kg)+aripiprazole (1 mg/kg) given 45 min prior to testing restored SI deficits to normal levels, suggesting efficacy for treatment resistant negative symptoms. The statistical test data are given in FIG. 7.

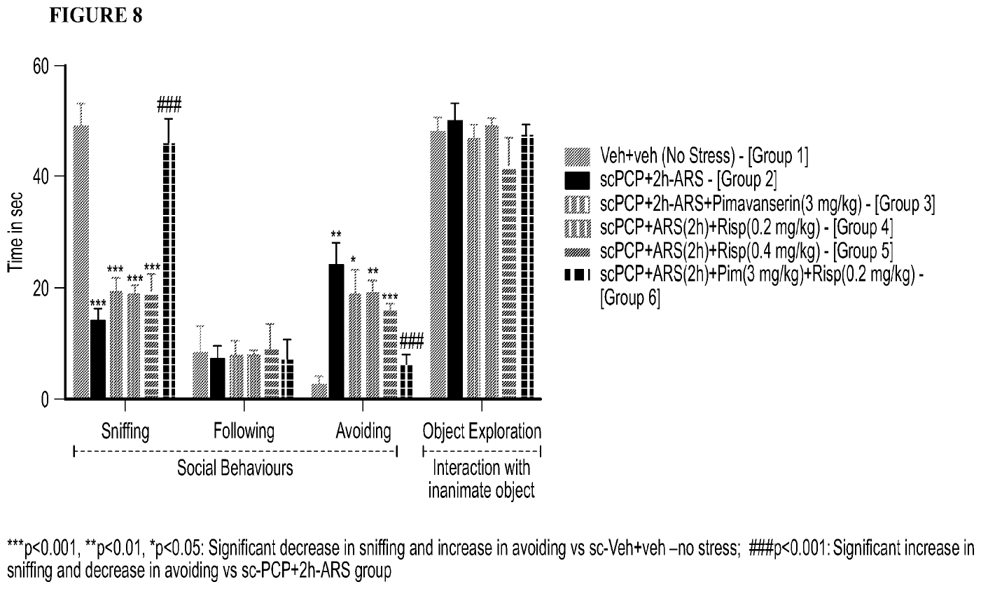

To confirm a treatment-resistant model of negative symptoms of schizophrenia that could only be rescued by combining pimavanserin with an atypical APD, the dose of risperidone administered to scPCP and ARS-treated mice was doubled to 0.4 mg/kg (FIG. 8). Even this higher dose of risperidone was ineffective, as was 0.2 mg/kg risperidone and 3 mg/kg pimavanserin alone. However, the combination of 0.2 mg/kg risperidone and 3 mg/kg pimavanserin significantly reduced avoiding behavior, and significantly increased sniffing behavior compared to scPCP and ARS-treated mice that received vehicle. The plasma levels of risperidone were 5.4±0.1, 5.3±0.6, and 8.0±3.3 in the 0.2 mg/kg risperidone, 0.2 mg/kg risperidone plus 3 mg/kg pimavanserin, and 0.4 mg/kg risperidone groups, respectively, confirming that the augmenting effects of pimavanserin were not due to a pharmacokinetic effect of pimavanserin on risperidone drug levels.

This data establish the combination of scPCP and 2 h ARS as a model of treatment resistance in social interaction and demonstrates that the addition of pimavanserin to a standard dose of an atypical APD can rescue the deficit in this model of negative symptoms. Thus, pimavanserin may be an effective adjunct to some atypical APDs for the treatment of negative symptoms in patients who have failed to respond to an adequate trial of these atypical APDs.

### Example 4—Phase 2, Randomized, Double-Blind, Placebo-Controlled Study to Evaluate the Efficacy and Safety of Pimavanserin as Adjunctive Treatment for the Negative Symptoms of Schizophrenia

This study is conducted as a Phase 2, 26-week, randomized, double-blind, placebo-controlled, multicenter, multinational outpatient study in subjects with schizophrenia who have predominant negative symptoms while on adequate treatment with an antipsychotic. Schizophrenia is defined by the Diagnostic and Statistical Manual of Mental Disorders, Fifth Edition (DSM-5), confirmed by a customized module of the Structured Clinical Interview for DSM-5, Clinical Trials Version (SCID-5-CT). This study enrolls approximately 403 subjects with predominant negative symptoms of schizophrenia. In the study, patients were randomized to receive either pimavanserin or placebo added to their current background antipsychotic treatment for 26 weeks in duration. At baseline all patients were started on a daily dose of 20 mg of pimavanserin or placebo with dose adjustments allowed up to 34 mg or down to 10 mg during the first eight weeks of treatment. Following week eight no further dose modifications were allowed for the remaining 18 weeks in the study.

The daily dose of the main antipsychotic must remain stable and may not be changed from Screening through the duration of the study. Subjects will participate in the study for up to 34 weeks, including a Screening Period of up to 4 weeks, a 26-week Treatment Period, and a 4-week safety follow-up (telephone call) for those subjects who discontinue prematurely from the study or who do not enroll in the 52-week, open-label extension study. At the Weeks 2, 4, and 8 visits, the daily dose of pimavanserin may remain at 20 mg or it may be either increased to 34 mg (for symptom improvement) or decreased to 10 mg daily (if the 20 mg dose is not well tolerated). After the Week 8 visit, no study drug dose changes may be made. Clinic visits occurring after Baseline will be conducted at Weeks 2, 4, 8, 14, 20, and 26 (End-of-Study [EOS]/Early Termination [ET] visit).

### Subject Inclusion Criteria

Male or female, age from 18 to 55 at the time of Screening are included in this study. Subjects must have a diagnosis of schizophrenia according to Diagnostic and Statistical Manual of Mental Disorders—Fifth Edition (DSM-5) criteria (confirmed using a customized module of the Structured Clinical Interview for DSM-5, Clinical Trials Version [SCID-5-CT]) made ≥1 year prior to randomization.

Subjects must have Score≥20 on the sum of the 7 PANSS Marder negative factor items at Screening and Baseline AND Score≥4 on at least 3, or ≥5 on at least 2, of the 7 PANSS Marder negative factor items.

Subjects must have Score≤22 on the sum of the 8 PANSS Marder positive factor items AND PANSS score where no more than two of the following items have a score of 4 and none of the following items has a score≥5 at both Screening and Baseline: P1 (delusions); P3 (hallucinatory behavior); P6 (suspiciousness/persecution).

Subjects must have a Clinical Global Impression of Schizophrenia Scale-Severity (CGI-SCH-S) for the negative symptoms of schizophrenia score≥4 (moderately ill or worse) at Screening and Baseline.

CGI-SCH-S scale was adapted from the CGI scale and designed to assess positive, negative, depressive and cognitive symptoms in schizophrenia.

Subjects must have been treated with an adequate dose of an antipsychotic within the dose range recommended according to the local Prescribing Information for at least 8 weeks prior to Screening and remaining at the same dose during the Screening Period.

The antipsychotic with which the subject is being treated must be one of the antipsychotics selected from: Aripiprazole (Aripiprazole long-acting injectables, Abilify Maintena®), Aristada®, Asenapine, Brexpiprazole, Cariprazine, Lurasidone, Olanzapine, Risperidone, Risperidone long-acting injection.

### Subject Exclusion Criteria

Subjects were excluded from this study if Subjects are treated with 2 or more antipsychotics, for any indication, within 8 weeks prior to Screening, are taking a medication or drug or other substance that is prohibited according to this study including medications that prolong the QT interval, strong cytochrome P450 (CYP) 3A4 enzyme (CYP3A4) inhibitors and inducers, or have known family or personal history or symptoms of long QT syndrome.

### Methods

The test products are pimavanserin 10 mg and 17 mg tablets or matching placebo. Daily doses of pimavanserin to be studied are 10 mg (provided as 1×10 mg pimavanserin tablet and 1× matching placebo); 20 mg (2×10 mg pimavanserin tablets); or 34 mg (2×17 mg pimavanserin tablets); or matching placebo (2× placebo tablets); delivered by mouth. Seventeen (17) mg of the active moiety is dosed as 20 mg of the salt pimavanserin tartrate; 10 mg of the active moiety is dosed as 11.8 mg of the salt pimavanserin tartrate. The duration of participation for individual subjects will be up to approximately 34 weeks.

Primary Efficacy Endpoint is the change from Baseline to Week 26 in the Negative Symptom Assessment-16 (NSA-16) total score.

Secondary Endpoints Key Secondary Endpoint is the change from Baseline to Week 26 in the Personal and Social Performance Scale (PSP) score.

Other Secondary Endpoints are the change from Baseline to Week 26 in the CGI-SCH-S of negative symptoms score, Clinical Global Impression of Schizophrenia Scale-Improvement (CGI-SCH-I) of negative symptoms score at Week 26, the change from Baseline to Week 26 in the Positive and Negative Syndrome Scale (PANSS) total score, the change from Baseline to Week 26 in PANSS subscores, the change from Baseline to Week 26 in Brief Assessment of Cognition in Schizophrenia (BACS) score, the change from Baseline to Week 26 in 10-item Drug Attitude Inventory (DAI-10) score, and the change from Baseline to Week 26 in Karolinska Sleepiness Scale (KSS) score.

The Brief Assessment of Cognition in Schizophrenia (BACS) is an instrument for assessing the aspects of cognition found to be most impaired and most strongly correlated with outcome in patients with schizophrenia.

Baseline characteristics were very similar across the two treatment arms. The most prevalent background antipsychotics in the study included risperidone at 39%, aripiprazole at 33%, and olanzapine at 28%. The average age of patients in the study was 37.2 years and they had been on background antipsychotic medication for an average of 27 months.

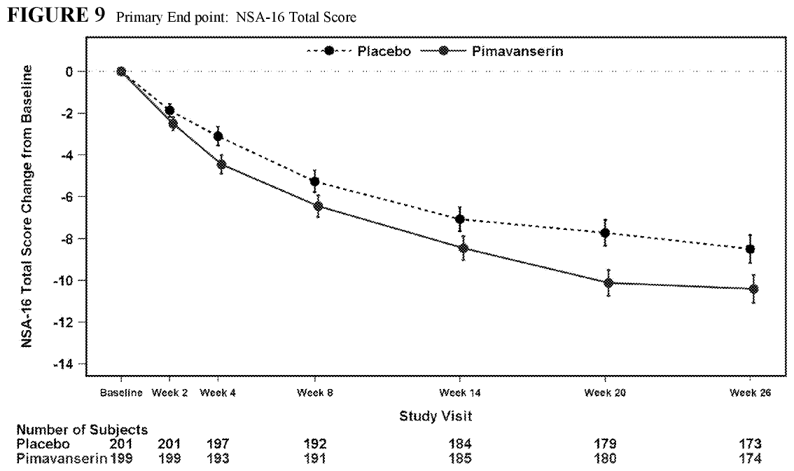

Pimavanserin met the primary endpoint by demonstrating a statistically significant reduction on the Negative Symptom Assessment-16 (NSA-16) total score compared to the placebo (−10.4 vs. −8.5; p=0.043; effect size=0.21). 53.8% of patients in the study remained on the highest dose level of to 34 mg (n=107). A greater reduction in the NSA-16 total score compared to placebo was observed in the patients who received the 34 mg fixed dose after week 8 (−11.6 vs. −8.5; unadjusted p=0.0065, effect size=0.34). See FIG. 9. In this study, pimavanserin did not show statistically significant difference from placebo on the key secondary endpoint, the Personal and Social Performance Scale (8.1 vs. 8.1; p=0.9806).

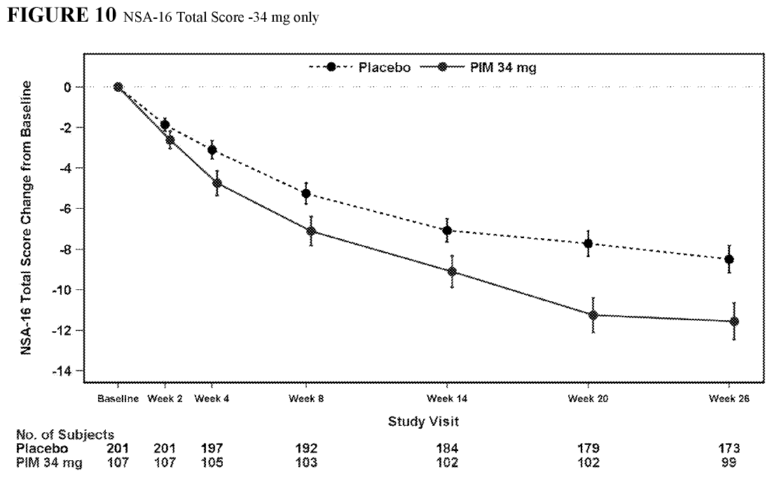

An important finding from the study was the greater effect observed with the 34 mg dose. FIG. 10 is a graph showing a consistent and positive improvement on the primary endpoint. The p-value for this post-hoc analysis is 0.0065 with a more meaningful effect size of 0.34.

Pimavanserin was well tolerated with similar rates of adverse events between adjunctive pimavanserin, 40%, and adjunctive placebo, 35%. The adjunctive use of pimavanserin did not result in clinically significant differences in vital signs, weight, metabolic syndrome, and extrapyramidal symptoms compared to adjunctive placebo.

Exploratory Endpoints are the change from Baseline to Week 26 in PANSS Marder factor scores, the change from Baseline to Week 26 in Calgary Depression Scale for Schizophrenia (CDSS) score, and the change from Baseline to Week 26 in 36-item Short Form Health Survey (SF-36) score.

The SF-36 is a patient-reported survey of health status assessing 8 health concepts: 1) limitations in physical activities because of health problems; 2) limitations in social activities because of physical or emotional problems; 3) limitations in usual role activities because of physical health problems; 4) bodily pain; 5) general mental health (psychological distress and well-being); 6) limitations in usual role activities because of emotional problems; 7) vitality (energy and fatigue); and 8) general health perceptions.

### Safety

Safety will be evaluated by analyses of adverse events, vital signs, ECGs, physical examination results, and clinical laboratory tests (including urinalysis), and the Abnormal Involuntary Movement Scale (AIMS), the Barnes Akathisia Rating Scale (BARS), the Simpson-Angus Extrapyramidal Side Effects Scale (SAS), and the Columbia Suicide Severity Rating Scale (C-SSRS).

### Pharmacokinetic Assessments

At each predefined timepoint, PK samples will be obtained for measurement of concentrations of pimavanserin, its metabolite AC-279, and the main antipsychotic. When possible, an additional PK sample will be collected from subjects who experience a serious adverse event (SAE) or an adverse event (AE) leading to discontinuation, as soon as possible after the occurrence of that event. For all PK samples (scheduled and unscheduled), the dates and times of administration of the last 3 doses of both study drug and the main antipsychotic should be recorded. For samples collected from subjects who experience an SAE or an AE leading to discontinuation, the date and time of the last dose prior to the SAE or AE should also be recorded. Pimavanserin plasma concentration data will remain blinded until the unblinding of the clinical database at the end of the study. Pharmacokinetic Endpoints: Plasma concentration of pimavanserin, AC-279, the main antipsychotic and Pimavanserin pharmacokinetic parameters using a population pharmacokinetic approach, and PK/PD using appropriate PK/PD analysis methods.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Certain features that are described in this specification in the context or separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A method of treating the loss of normal functions in a patient suffering from schizophrenia, wherein the patient has had a partial but inadequate response to an antipsychotic treatment, and is being administered the antipsychotic treatment, comprising orally administering once daily an effective amount of pimavanserin to the patient.

2. The method of claim 1, wherein the patient is not resistant to antipsychotic treatment.

3. The method of claim 1 or 2, wherein the loss of normal function is a negative symptom of schizophrenia.

4. The method of claim 3, wherein the negative symptom is at least one of: emotional blunting, emotional withdrawal, poor rapport, passive/apathetic social withdrawal, difficulty in abstract thinking, lack of spontaneity and flow of conversation, and stereotyped thinking.

5. The method of claim 1 or 2, wherein the loss of normal function is an aspect of sleep disturbance associated with schizophrenia.

6. The method of any one of claims 1-5 wherein after 6 weeks of administering pimavanserin, the patient improves on the negative syndrome subscale of PANSS compared to baseline.

7. The method of any one of claims 1-6, wherein after 6 weeks of administering pimavanserin, the patient improves on the PANSS Marder Negative factor score compared to baseline.

8. The method of any one of claims 1-7, wherein after 6 weeks of administering pimavanserin, the patient improves on the Karolinska Sleepiness Scale.

9. A method of treating or diminishing a negative symptom of schizophrenia in a patient having a partial but inadequate response to an antipsychotic alone, comprising administering an effective amount of pimavanserin to the patient and continuing administration of the antipsychotic.

10. A method of treating a patient having a partial but inadequate response to an antipsychotic treatment for schizophrenia, comprising:
continuing to administer an effective amount of the antipsychotic; and
administering an effective amount of pimavanserin, thereby treating loss of normal functions in the patient suffering from schizophrenia.

11. The method of claim 10, wherein the loss of normal function is a negative symptom of schizophrenia.

12. The method of any one of claims 1-11, wherein the antipsychotic is selected from the group consisting of quetiapine, clozapine, aripiprazole, asenapine, cariprazine, brexpiprazole, lurasidone, olanzapine, and risperidone.

13. The method of any one of claims 1-12, comprising administering about 10 mg to about 34 mg daily pimavanserin.

14. The method of any one of claims 1-13, comprising administering daily 10 mg or 34 mg pimavanserin.

15. The method of any one of claims 1-13, wherein administering an effective amount of pimavanserin comprises:
administering about 20 mg of pimavanserin daily for one to three weeks or more; and then
administering about 10 mg or about 34 mg pimavanserin daily.

16. A method of treating or diminishing a negative symptom of schizophrenia in a patient being administered an antipsychotic, comprising additionally administering an effective amount of pimavanserin to the patient and continuing administration of the antipsychotic.

17. The method of claim 16, wherein the negative symptom is at least one of: emotional blunting, emotional withdrawal, poor rapport, passive/apathetic social withdrawal, difficulty in abstract thinking, lack of spontaneity and flow of conversation, and stereotyped thinking.

18. The method of 16 or 17 wherein after 6 weeks of administering pimavanserin, the patient improves on the negative syndrome subscale of PANSS compared to baseline.

19. The method of any one of claims 16-18, wherein after 6 weeks of administering pimavanserin, the patient improves on the PANSS Marder Negative factor score compared to baseline.

20. The method of any one of claims 16-19, wherein after 6 weeks of administering pimavanserin, the patient improves on the Karolinska Sleepiness Scale.

21. The method of any one of claims 1-20, wherein after 26 weeks of administering pimavanserin, the patient improves on the PANSS Marder Negative factor score compared to baseline.

22. The method of any one of claims 1-21, wherein after 26 weeks of administering pimavanserin, the patient improves on the Karolinska Sleepiness Scale.

23. The method of any one of claims 1-22, wherein after 26 weeks of administering pimavanserin, the patient improves on the Negative Symptom Assessment-16 total score as compared to baseline.

24. The method of any one of claims 1-23, wherein after 26 weeks of administering pimavanserin, the patient improves on the Personal and Social Performance Scale (PSP) score as compared to baseline.

25. The method of any one of claims 1-24, wherein after 26 weeks of administering pimavanserin, the patient improves on the Negative Symptom Assessment (NSA-16) total score, Clinical Global Impression of Schizophrenia Scale-Severity (CGI-SCH-S) for the negative symptoms of schizophrenia score, the Positive and Negative Syndrome Scale (PANSS) score, the Brief Assessment of Cognition in Schizophrenia (BACS) score, or the 10-item Drug Attitude Inventory (DAI-10) score as compared to baseline.

26. A method of adjunctively treating negative symptoms of schizophrenia in need thereof, wherein the patient is currently being administered an antipsychotic, comprising additionally administering an effective amount of pimavanserin daily, wherein after 26 weeks of administration, the patient demonstrates a statistically significant reduction on the Negative Symptom Assessment-16 (NSA-16) total score.

27. The method of claim 26, wherein the effective amount is 10 mg to 34 mg of pimavanserin daily.

28. The method of claim 26 or 27, wherein the effective amount is 34 mg of pimavanserin daily.

29. The method of claim 26 or 27, wherein the effective amount is 10 or 20 mg of pimavanserin daily.

30. The method of claim 26 or 27, wherein administering comprises administering an initial dose of 20 mg daily of pimavanserin for 1 day, 1 week, or 1-8 weeks, and then administering 34 mg daily for at least 18 weeks.

31. The method of any one of claims 26-30, where in the antipsychotic is one of aripiprazole, asenapine, cariprazine, brexpiprazole, lurasidone, olanzapine or risperidone.

32. A method of treating psychosis secondary to neurodegenerative disorders or major depressive disorder, in a patient in need thereof, comprising administering about 20 mg of pimavanserin daily for one to three weeks or more; and then administering about 10 mg or about 34 mg pimavanserin daily.

33. The method of claim 32, wherein psychosis secondary to neurodegenerative disorders is dementia related psychosis.
